(19) United States
(12) Patent Application Publication
(10) Pub. No.: US 2023/0170070 A1
(43) Pub. Date: Jun. 1, 2023

(54) SYSTEM AND METHOD FOR PROVIDING FERTILITY ENHANCING DIETARY RECOMMENDATIONS IN INDIVIDUALS WITH SPERM CONDITIONS

(71) Applicant: SOCIETE DES PRODUITS NESTLE S.A., Vevey (CH)

(72) Inventors: MYRIAM AFEICHE ZEHIL, Lausanne 26 (CH); FLORENT DUDAN, St-Sulpice (CH); FABIO MAINARDI, Lausanne (CH); ROKO PLESTINA, Morges (CH)

(21) Appl. No.: 17/997,274

(22) PCT Filed: Apr. 27, 2021
(86) PCT No.: PCT/EP2021/060885

§ 371 Date: Oct. 27, 2022

(30) Foreign Application Priority Data
Apr. 29, 2020 (EP) 20172023.2

(51) Int. Cl. G16H 20/60; G16H 20/10

(52) U.S. Cl. CPC G16H 20/60; G16H 20/10; G16H 10/20

(57) ABSTRACT

The present invention presents new and innovative methods and systems for personalized, realtime diet and lifestyle recommendations for users that are seeking to improve their own fertility. In a preferred embodiment, the present invention relates to novel methods and systems for personalized dietary recommendations for improving fertility in individuals, especially men, with sperm conditions such as reduced sperm count, impaired sperm motility, abnormal sperm morphology and reduced sperm quantity.

### FIELD OF THE INVENTION

The present invention presents new and innovative methods and systems for personalized, real-time diet and lifestyle recommendations for users that are seeking to improve their own fertility.

In a preferred embodiment, the present invention relates to novel methods and systems for personalized dietary recommendations for improving fertility in individuals, especially men, with various abnormal sperm conditions such as reduced sperm count, impaired sperm motility, abnormal sperm morphology and reduced sperm quantity.

### BACKGROUND TO THE INVENTION

Infertility affects about 8-12% of reproductive-aged couples worldwide. An initial screening evaluation of the male partner of an infertile couple is indicated when pregnancy has not occurred after 12 months of unprotected intercourse or after 6 months of failure to conceive when the female partner is greater than 35 years old. In 50% of cases, a male factor of infertility underlies the problem, but in about 30% of these cases the etiology of male infertility remains unknown. Unlike unexplained male infertility with its normal semen parameters, idiopathic male infertility is diagnosed in the presence of altered semen characteristics without an identifiable cause and the absence of female factor infertility.

Semen analysis remains the clinical standard for assessing male fecundity and related impairments, including hormone production, and key components, such as sperm concentration, motility, and morphology, are reported to be capable of classifying men by fertility potential. While the World Health Organization (WHO) publishes reference values for semen parameters, men with semen parameters outside the reference range may still be fertile, and, equally, men with semen parameters within normal range may be subfertile. While semen quality parameters are not perfect proxies of men's fertility but they commonly accepted clinical standards of for male fertility.

Sperm count refers to the average number of sperm present in one sample of semen. Fertility doctors assess sperm count during routine semen analyses and consider it to be an important factor for fertility. Based on the most recent World Health Organization (WHO) guidelines, experts consider a healthful sperm count to be 15 million per milliliter (ml), or at least 39 million per ejaculate. Doctors consider a sperm count under 15 million per ml to be low, and it may cause fertility issues. Overall, health professionals believe that factors that influence testosterone levels have also have a significant impact on sperm number and quality.

Healthy sperm production both in terms of motility, morphology and quantity are important factors influencing fertility and subsequent chances of conception.

Men can be classified as “normozoospermic” meaning that there are no detectable anomalies in the spermiogram. Anomalies in sperm may be defined in terms of sperm motility, morphology or number.

“Asthenozoospermic” are defined as having less than 40% sperm motility or less than 32% with progressive sperm motility.

“Teratozoospermic” are defined as having abnormal sperm morphology with less than 30% morphologically normal sperm.

“Oligozoospermic” are defined as having low sperm concentration with less than 20×10⁶/mL concentration in semen.

Other measures of sperm function may exist, such as measurement of markers of sperm DNA integrity and markers of sperm oxidative stress.

Causes of male infertility which influence healthy sperm production include hormonal imbalance, varicocele, diseases, genetic factors, lifestyle factors (such as obesity, advanced paternal age, alcohol, exercise, smoking, drug addiction, increased sedentary activity, lower physical activity), environmental contaminants, and secular changes in diet quality. Diet and lifestyle are modifiable factors that have been shown to improve semen quality parameters and most importantly pregnancy rates in the female partner.

As men are delaying the age at which they would like to remain fertile, they are at increasing risk of having poor quality and quantity of sperm. This is exacerbated in conditions of obesity. Therefore, there is a need to find ways to improve fertility in men at risk of conditions which affect both quality and quantity of sperm produced.

At present, dietary recommendations for men with conditions affecting sperm are often too general as studies on fertility and diet often investigate only single nutrients at a time or food groups without putting them in the context of an entire diet over a day or entire meal and they do not provide recommended intake amounts to be consumed per day or for each specific, different medical condition which affects fertility, such as sperm health conditions. In addition there are plenty of antioxidants on the market claiming to improve semen quality parameters, but the doses and durations need to be verified in clinical trials.

The present invention addresses the deficiencies in the state of the art by providing new and innovative methods and systems for personalized, real-time diet and lifestyle recommendations for individual users.

In particular, the present invention addresses the specific condition of enhancing fertility and conception in individuals with sperm conditions, by providing a novel, consolidated dietary recommendations which combine:
specific dietary components recommended to be consumed daily
specific dietary intake amounts
specific recommendations on avoiding certain dietary components
specific recommendations on lifestyle components.

### SUMMARY OF THE INVENTION

The present invention presents novel and innovative methods and systems for personalized, real-time diet and lifestyle recommendations for users that are seeking to improve their own fertility.

In several embodiments, a method and system is provided that includes requesting and receiving a plurality of user attributes, comparing the plurality of user attributes to a corresponding plurality of evidence-based fertility benchmarks, determining a plurality of fertility support opportunities based on the plurality of user attributes and the comparison to the corresponding plurality of evidence-based fertility benchmarks, identifying a plurality of fertility enhancing recommendations based on the plurality of fertility support opportunities, and presenting at least one of the plurality of fertility enhancing recommendations.

In a preferred embodiment of the invention, the system and method present fertility enhancing recommendations are for increasing fertility in a male individual with sperm conditions such as reduced sperm count, reduced sperm motility, abnormal morphology of sperm or reduced sperm viability.

### DESCRIPTION OF FIGURES

FIG. 1 illustrates a system according to an embodiment of the present invention.

FIG. 2 illustrates system components according to exemplary embodiments of the present invention.

FIG. 3 illustrates system components according to an exemplary embodiment of the present invention.

FIG. 4 illustrates an example plurality of dietary recommendations for a user according to an exemplary embodiment of the present invention.

FIG. 5 illustrates a method according to an embodiment of the present invention.

FIGS. 6A and 6B illustrate a method according to an embodiment of the present invention.

### DETAILED DESCRIPTION OF THE INVENTION

To promote user fertility, it may be useful to provide a customized diet and lifestyle plan for users interested enhancing fertility. Therefore, a customized, integrated approach is necessary to provide maximum benefit to enhance chances of fertility and conception.

In several embodiments of the invention, the customized diet and lifestyle plan is individualized to male users with sperm conditions.

Sperm count during routine semen analyses is considered to be an important factor for fertility.

Sperm count refers to the average number of sperm present in one sample of semen. A healthy sperm count is considered to be 15 million sperm per milliliter (ml), or at least 39 million per ejaculate. If the sperm count is below 15 million per ml, there may be resulting fertility issues.

In addition, factors that influence testosterone levels have a significant impact on sperm number and quality. Low testosterone levels below normal range of around 270 to 1,070 nanograms per deciliter (ng/dL) can result in fertility conditions. Certain medical conditions including inherited genetic disorders, infections, and tumors can also impact sperm count.

Semen Analysis

According to the World Health Organisation guidelines set in 2010, WHO Laboratory Manual for the Examination and Processing of Human Semen, 5th ed.; WHO Press: Geneva, Switzerland, 2010; ISBN 978-924-154-778-9, a semen analysis looks at several factors for determining normal fertility based on the following criteria:

Concentration—15 million per ml or 39 million in the entire sample.

Volume—1.5 ml on average.

Motility—At least 32% of progressively motile sperm.

Viability—Greater than 58% living sperm per sample.

Morphology—At least 4% normal shaped sperm.

Liquefaction—Semen stays in liquid form for 60 minutes after ejaculation.

Viscosity—Too thick of a sample could indicate a problem with the prostate.

Color—Opalescent, slightly yellow color.

pH level—above 7.1*Lower values might indicate azoospermia, or no level of measurable sperm in the semen.

Leukocytes—Less than 1 million per ml of sample. Greater number could indicate infection.

Antisperm antibodies—Less than 50%; anything greater suggests an immune disorder.

In a preferred embodiment of the invention, the customized diet and lifestyle plan is individualized to male users with sperm conditions, such as low sperm count below 15 million sperm per milliliter.

In a preferred embodiment of the invention, the customized diet and lifestyle plan is individualized to male users with sperm conditions, such as having less than 40% sperm motility or less than 32% with progressive sperm motility.

In a preferred embodiment of the invention, the customized diet and lifestyle plan is individualized to male users with sperm conditions, such as having abnormal sperm morphology with less than 30% morphologically normal sperm.

In a preferred embodiment of the invention, the customized diet and lifestyle plan is individualized to male users with sperm conditions, such as low sperm concentration with less than 20×10⁶/mL concentration in semen.

In a preferred embodiment of the invention, the customized diet and lifestyle plan is individualized to male users with sperm conditions such as low testosterone below 270 to 1,070 nanograms per deciliter (ng/dL).

In another preferred embodiment, the customized diet and lifestyle plan is individualized to male users with sperm conditions who are additionally obese and may require a restricted caloric intact to lose weight. Adult Body Mass Index (BMI) is a person's weight in kilograms divided by the square of height in meters.

If BMI is less than 18.5, one is considered within the underweight range.

If BMI is 18.5 to <25, one is considered within the normal range.

If BMI is 25.0 to <30, one is considered within the overweight range.

If your BMI is 30.0 or higher, one is considered within the obese range.

In one preferred embodiment of the invention, the customized diet and lifestyle plan is individualized to male users with sperm conditions associated with a BMI 30.0 or greater. Such a customized plan may include daily caloric restriction to lose weight as well as an exercise plan.

In any of the above conditions of sperm conditions, the methods and systems of the invention for providing individualized recommendations may be beneficial.

In several embodiments of the invention, the recommendations are personalized based on information from an individual regarding certain medical conditions or diseases and the current status of conception to compare with a historical evidence-based fertility database to generate recommended diet and lifestyle options that will help improve the patient's fertility based on the provided information.

An example system may be beneficial if it can provide user support throughout the entire conception journey, from the early stages of planning to the final stages of conception. As such, this example system would be useful should it provide constant, around the clock access to both virtual and personal fertility, lifestyle, nutrition, and exercise coaches. Furthermore, an example system may provide recommendations to manage anxiety, reduce stress, or provide specific supplementation, all of which are also linked to a user's fertility. The system may provide different recommendations for the male and female individuals in the couple that wish to conceive.

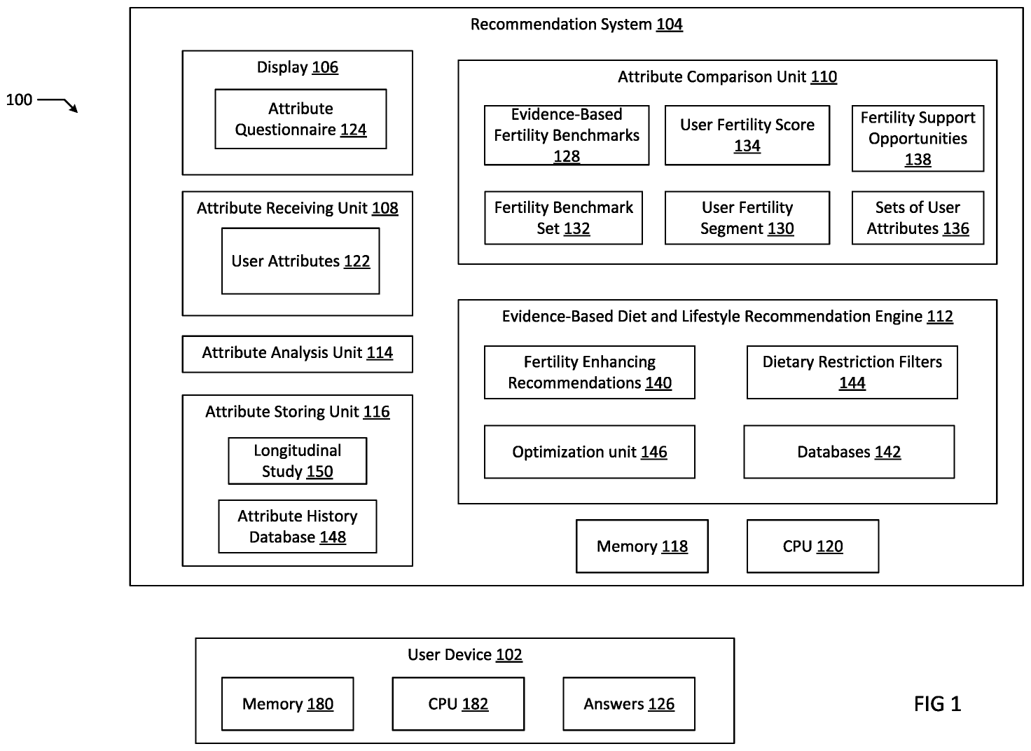

FIG. 1 illustrates a system 100 according to an embodiment of the present disclosure. The system 100 includes a user device 102 and a recommendation system 104. The user device 102 may be implemented as a computing device, such as a computer, smartphone, tablet, smartwatch, or other wearable through which an associated user can communicate with the recommendation system 104. The user device 102 may also be implemented as, e.g., a voice assistant configured to receive voice requests from a user and to process the requests either locally on a computer device proximate to the user or on a remote computing device (e.g., at a remote computing server).

The recommendation system 104 includes one or more of a display 106, an attribute receiving unit 108, an attribute comparison unit 110, an evidence-based diet and lifestyle recommendation engine 112, an attribute analysis unit 114, an attribute storing unit 116, a memory 118, and a CPU 120. Note, that in some embodiments, a display 106 may additionally or alternatively be located within the user device 102. In an example, the recommendation system 104 may be configured to receive a request for a plurality of fertility enhancing recommendations 140. For example, a user may install an application on the user device 102 that requires the user to sign up for a recommendation service. By signing up for the service, the user device 102 may send a request for the fertility enhancing recommendations 140. In a different example, the user may use the user device 102 to access a web portal using user-specific credentials. Through this web portal, the user may cause the user device 102 to request fertility enhancing recommendations from the recommendation system 104.

In another example, the recommendation system 104 may be configured to request and receive a plurality of user attributes 122. For example, the display 106 may be configured to present an attribute questionnaire 124 to the user. The attribute receiving unit 108 may be configured to receive the user attributes 122. In one example, the attribute receiving unit 108 may receive a plurality of answers 126 based on the attribute questionnaire 124, and based on the plurality of answers, determine the plurality of user attributes 122. For example, the attribute receiving unit 108 may receive answers to the attribute questionnaire 124 suggesting that the diet of the user is equivalent to the recommended dietary allowance (“RDA”) and then determine the user attributes 122 to be equivalent to the RDA. In another example, the user device attribute receiving unit 108 may directly receive the user attributes 122 from the user device 102.

In another example, the attribute receiving unit 108 may be configured to receive the test results of a home-test kit, the results of a standardized health test administered by a medical professional, the results of a self-assessment tool used by the user, or the results of any external or third party test. Based on the results from any of these tests or tools, the attribute receiving unit 108 may be configured to determine the user attributes 122. For example, this may be measurements of the sperm count of the user which may be compared to standardized sperm counts.

The recommendation system 104 may be further configured to compare the plurality of user attributes 122 to a corresponding plurality of evidence-based fertility benchmarks 128. For example, the attribute comparison unit 110 may be configured to determine a user fertility segment 130.

In a preferred example, the user fertility segment could be much more specific. For example, the user fertility segment could be a user with sperm conditions.

Furthermore, the attribute comparison unit 110 may be further configured to determine a fertility benchmark set 132 based on the user fertility segment 130. For example, if the attribute comparison unit 110 determines that a user falls into the sperm condition user fertility segment 130, based on the plurality of user attributes 122, the attribute comparison unit 110 may select a fertility benchmark set 132 that has been created and defined according to the specific needs of a user undergoing a particular medical treatment or weight loss treatment in the case of an obese subject.

The comparison unit 110 may be further configured to select, from this determined fertility benchmark set 132, the evidence-based fertility benchmarks 128 and compare the now selected evidence-based fertility benchmarks 128 to each of the corresponding user attributes 122. For example, when the fertility benchmark set 132 has been determined, in response to the determination, the attribute comparison unit 110 may compare a user attribute 122 that represents the user's vitamin E intake to an evidence based fertility benchmark 128 that represents a benchmark vitamin E intake, determining whether the user is below, at, or above the benchmark vitamin E intake. Though this example is based on a concrete, numerical comparison, another example of a benchmark comparison may be qualitative and different depending on a person. For example, a user attribute 122 may indicate that the user is currently experiencing higher than normal levels of stress. An example benchmark related to a user stress level may indicate that an average or low level of stress is desired and thus, the user attribute 122 indicating a higher level of stress is determined to be below that of the benchmark. As different users experience differing levels of stress, even under the same circumstances, such a comparison requires a customized approach.

In addition, during the comparison from the prior example, the attribute comparison unit 110 may be configured to determine a user fertility score 134 based on the comparison between the evidence-based fertility benchmarks 128 and the user attributes 122. For example, the attribute comparison unit 110 may determine a user fertility score of 95/100 if the user attributes 122 very nearly meet all or most of the corresponding evidence-based fertility benchmarks 128. In another example, a score may be represented through lettering grades, symbols, or any other system of ranking that allows a user to interpret how well their current attributes rate amongst benchmarks. This user fertility score 134 may be presented through the display 106.

The recommendation system 104 may be further configured to determine a plurality of fertility support opportunities 138 based on the plurality of user attributes 122 and the comparison to the corresponding plurality of evidence-based fertility benchmarks 128. In one example, the attribute comparison unit 110 may determine fertility support opportunities 138 for every user attribute 122 that does not meet the corresponding evidence-based fertility benchmark. In this example, a corresponding evidence-based fertility benchmark 128 may require a user have an intake of 1000 mg/day of Vitamin E, whereas the user attribute may indicate the user is only receiving 500 mg/day of Vitamin E. Therefore, the attribute comparison unit 110 may determine an increase in Vitamin E intake to be a fertility support opportunity 138.

In another example, the attribute comparison unit 110 may be configured to identify a first set of user attributes 136 comprised of each of the plurality of user attributes 122 that are below the corresponding one of the plurality of evidence-based fertility benchmarks 128 as well as identify a second set of user attributes 136 comprised of each of the plurality of user attributes 122 that are greater than or equal to the corresponding evidence-based fertility benchmarks 128. While the first set of user attributes 136 is determined similarly to the above given example, the second set of user attributes 136 differs in that, although the associated user does not appear to have a deficiency, there may be opportunities to support fertility by recommending the user maintain current practices or opportunities to further improve upon them. Accordingly, the recommendation system 104 may determine opportunities to support fertility based on which attributes 122 populate either sets 136.

The recommendation system 104 may be further configured to identify a plurality of fertility enhancing recommendations 140 based on the plurality of fertility support opportunities 138. For example, the evidence-based diet and lifestyle recommendation engine 112 may be configured to be cloud-based. The recommendation engine 112 may comprise one or more of a plurality of databases 142, a plurality of dietary restriction filters 144, and an optimization unit 146. Based on the plurality of opportunities 138, the recommendation engine 112 may identify the plurality of fertility enhancing recommendations 140 according to the one or more of plurality of databases 142, the dietary restriction filters 144, and the optimization unit 146.

In another example, the recommendation system 104 may be configured to provide continuous recommendations, based on prior user attributes. For example, the recommendation system 104 may comprise, in addition to the previously discussed elements, an attribute storing unit 116 and an attribute analysis unit 114. The attribute storing unit 116 may be configured to, responsive to the attribute receiving unit 108 receiving the plurality of user attributes 122, add the received user attributes 122 to an attribute history database 148 as a new entry based on when the plurality of user attributes 122 were received. For example, if user attributes 122 are received by the attribute receiving unit 108 on a first day, the attribute storing unit 116 will add the received user attributes 122 to a cumulative attribute history database 148 noting the date of entry, in this case the first day. Later, if user attributes 122 are received by the attribute receiving unit 108 on a second day, e.g. the next day, the attribute storing unit 116 will also add these new attributes to the attribute history database 148, noting that they were received on the second day, while also preserving the earlier attributes from the first day.

This attribute analysis unit 114 may be configured to analyze the plurality of user attributes 122 stored within the attribute history database 148, wherein analyzing the stored plurality of user attributes 122 comprises performing a longitudinal study 150. Continuing the earlier example, the attribute analysis unit 114 may perform a longitudinal study of the user attributes 122 from each of the first day, the second day, and every other collection of user attributes 122 found within the attribute history database 148. The evidence based diet and lifestyle recommendation engine 112 may be further configured to generate a plurality of fertility enhancing recommendations 140 based on at least the stored user attributes 122 found within the attribute history database 148 and the analysis performed by the attribute analysis unit 114.

In an embodiment, the attribute analysis unit 114 is further configured to repeatedly analyze the plurality of user attributes 122 stored within the attribute history database 148 responsive to the attribute storing unit 116 adding a new entry to the attribute history database 148, essentially re-analyzing all of the data within the attribute history database 148 immediately after new user attributes 122 are received. Similarly, the evidence based diet and lifestyle recommendation engine 112 may be further configured to repeatedly generate the plurality of fertility enhancing recommendations 140 responsive to the attribute analysis unit 114 completing an analysis, thereby effectively generating new fertility enhancing recommendations 140 that consider all past and present user attributes 122 each time a new set of user attributes 122 is received.

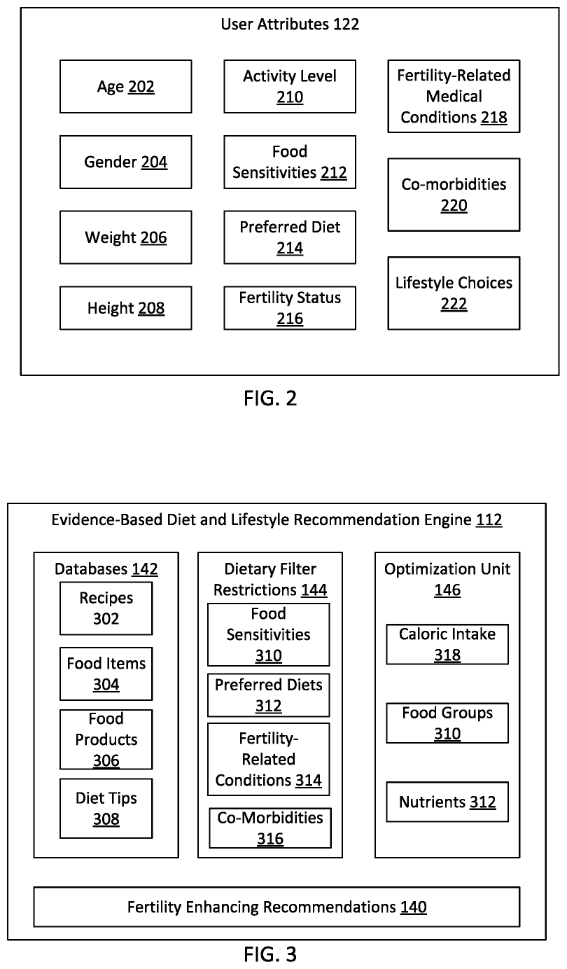

FIG. 2 illustrates an example database containing a plurality of user attributes 122. For example, the user attributes 122 may be populated by information regarding one or more of age 202, gender 204, weight 206, height 208, activity level 210, food sensitivities 212, preferred diet 214, fertility status 216, fertility-related medical conditions 218, co-morbidities 220, and lifestyle choices 222. Some examples of food sensitivities 212 include lactose, eggs, nuts, shellfish, soy, fish, and gluten sensitivities. Some non-limiting examples of a preferred diet 214 includes vegetarian, vegan, Mediterranean, kosher, halal, paleo, low carb, and low fat diets.

Some non-limiting examples of fertility-related medical conditions 218 include sperm conditions, prostate conditions, polycystic ovary syndrome, premature ovarian insufficiency, endometriosis, recurring pregnancy loss, undergoing IVF, semen abnormality, misuse of anabolic steroids and protein supplements, erectile dysfunction, hormonal imbalance, low testosterone, and prostate issues.

In several embodiments, the fertility-related medical condition 218 is a sperm-related condition.

In a preferred embodiment of the invention, the sperm related-condition is low sperm count below 15 million sperm per milliliter.

In a preferred embodiment of the invention, the sperm-related condition is low sperm motility with less than 40% sperm motility or less than 32% with progressive sperm motility.

In a preferred embodiment of the invention, the sperm-related condition is abnormal sperm morphology with less than 30% morphologically normal sperm.

In a preferred embodiment of the invention, the sperm-related condition is low sperm concentration with less than 20×106/mL concentration in semen.

In a preferred embodiment of the invention, the sperm-related condition is associated with low testosterone below 270 to 1,070 nanograms per deciliter (ng/dL).

In another preferred embodiment, the sperm-related condition is associated with an individual who is obese and may require a restricted caloric intact to lose weight. For example, an individual with sperm-related conditions associated with a BMI 30.0 or greater.

Some non-limiting examples of co-morbidities 220 include obesity, diabetes, high blood pressure, high cholesterol, celiac, and heartburn. Some non-limiting examples off lifestyle choices 222 may include sleeping habits such as the typical hours of sleep per night, stress attributes such as the level of stress currently experienced by the user or typical levels of stress experienced, whether the user smokes, the number of alcoholic drinks typically consumed, exercise frequency, or any other lifestyle choices 222 that may have a bearing on fertility.

FIG. 3 illustrates an example embodiment of an evidence-based diet and lifestyle recommendation engine 112. In an example embodiment, the evidence-based diet and lifestyle recommendation engine 112 comprises a plurality of databases 142, a plurality of dietary filter restrictions 144, and an optimization unit 146. The plurality of databases 142 may include a database comprised of one or more of recipes 302, food items 304, food products 306, and diet tips 308. The dietary filter restrictions 144 may comprise filters for one or more of food sensitivities 310, preferred diets 312, fertility-related conditions 314, and co-morbidities 316. The optimization unit 146 may contain optimization rules based on one or more of caloric intake 318, food groups 310, and specific nutrients 312.

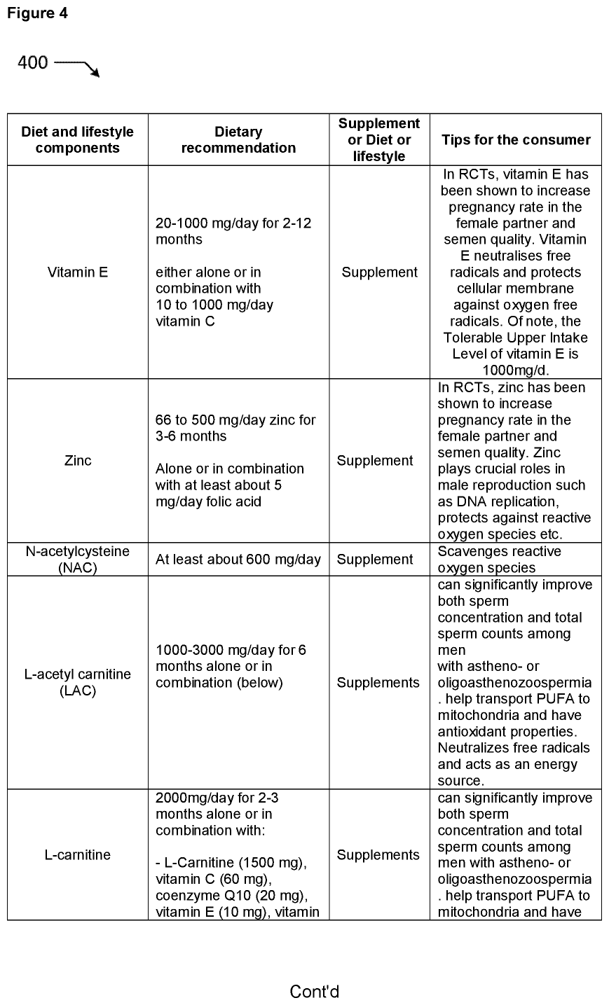

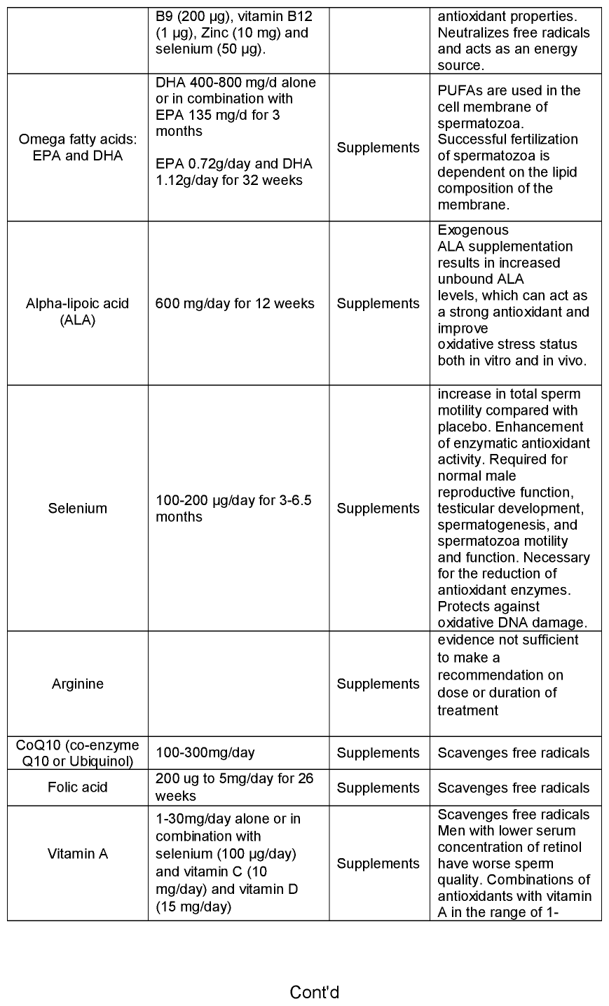

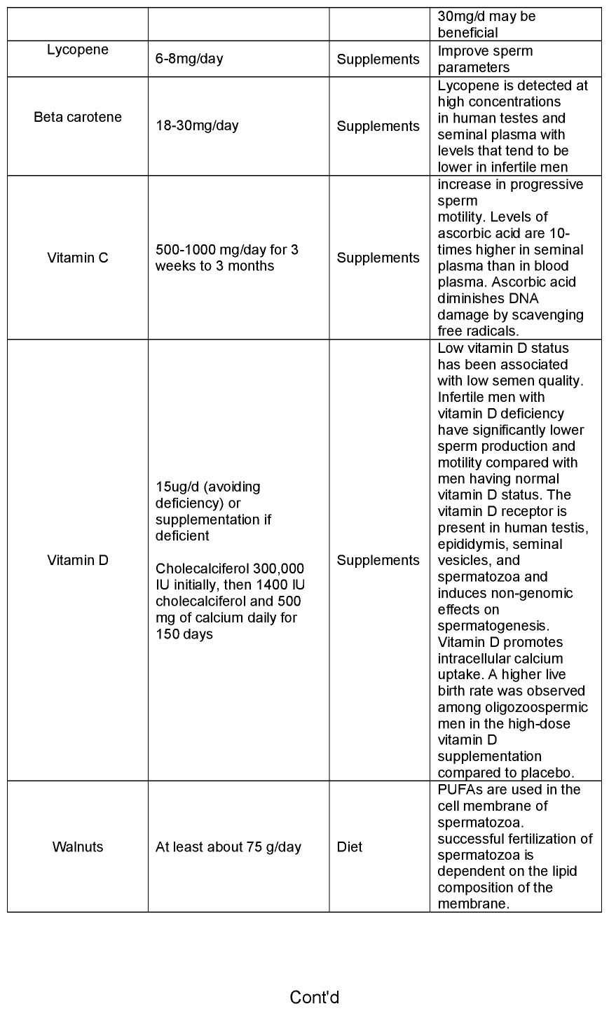

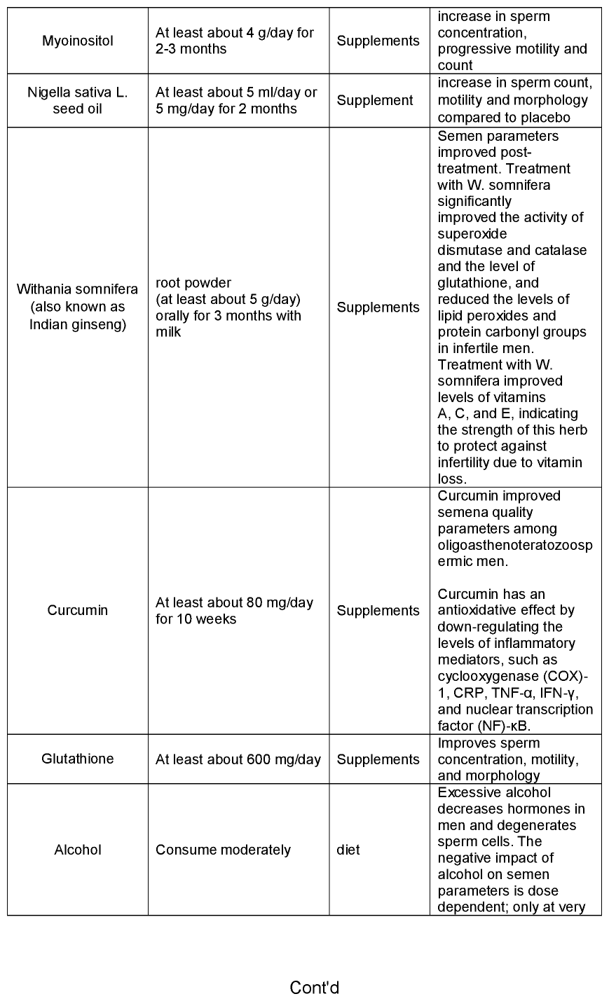

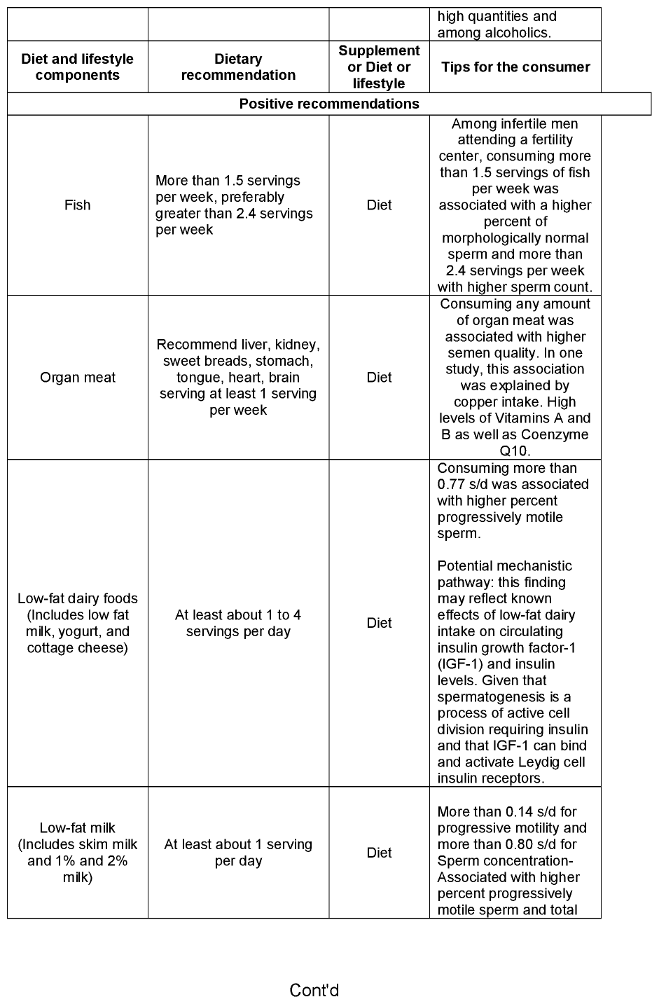

FIG. 4 illustrates an example plurality of dietary and lifestyle recommendations according to an exemplary embodiment of the present disclosure. This dietary recommendation example 400 details specific recommendations that may be presented to a user after a plurality of fertility enhancing recommendations 140 have been determined by the recommendation system 104. Specifically, example 400 details the fertility enhancing recommendations 140 as determined for a user that has a specific fertility-related medical condition 218. Specifically, example 400 represents the fertility enhancing recommendations as determined for a user with sperm conditions.

Other recommendations 140 may be to simply avoid, or increase, consumption of a particular food item. Similarly, the recommendations 140 may include recommendations to consume certain substances moderately or to prefer one substance over another. Though there are many different types of recommendations 140 found within the example 400, one should appreciate that any type of qualitative or quantitative recommendation may be made regarding these food items and nutrients.

Furthermore, the recommendation system 400 may generate fertility enhancing recommendations 140 that include lifestyle changes, such as altering activity level, increasing the number of hours of rest per night, taking action to reduce stress, or similar lifestyle-affecting actions. For example, a high level of stress may negatively affect a user's fertility. Such stress may derive from the relationship between the partners actively trying to conceive. Some example fertility enhancing recommendations 140 may include suggestions for methods by which the couple may decrease tensions in the relationship in order to alleviate stress. In another example, the fertility enhancing recommendations 140 may include recommendations to increase the amount of time a user rests, including sleeping habit recommendations. These recommendations may range from general recommendations, such as instruction to get more sleep, to more detailed recommendations, including specific exercise routines, specific diets and recipes, or suggested dates for visits to a medical professional.

In addition, in another embodiment, the fertility enhancing recommendations 140 generated by the recommendation system 104 may include specific recommendations for a product. For example, the recommendation system 104 may access a database containing information on a variety of supplements in the market. Then, based on its own analysis or through the use of third party research, the recommendation system 104 may analyze the different options for a specific supplement, such as Vitamin E, to determine that a specific supplement from a first brand, Brand A, is the most beneficial supplement as compared to other Vitamin E supplements available from a second, third, and fourth brand. Such an analysis may be conducted based on the quality of the supplements, the cost of the supplements, known side effects, method of manufacture, or any other factors that may distinguish the supplement provided by one brand from a supplement provided by another brand. The recommendation system 104 may provide similar recommendations as related to food items, such as a particular type or brand of apple, and any other category of product which may require the user to select one of multiple available options.

Supplements

In some embodiments of the invention, the recommendation system recommends specific dietary supplements for individuals with sperm conditions who would like to improve their fertility.

In preferred embodiments of the invention, the dietary supplements are selected from the group of:

Vitamin E

Vitamin E is a fat soluble vitamin with antioxidant properties. It is found naturally in some foods and added to others, and available as a dietary supplement. Alpha-tocopherol is the form that is recognized to meet human dietary requirements.

Zinc

Zinc is a mineral also known as an “essential trace element” because very small amounts of zinc are necessary for human health. Since the body does not store excess zinc, it must be consumed regularly as part of the diet. Common dietary sources of zinc include red meat, poultry, and fish.

N-Acetyl Cysteine

N-acetyl cysteine (NAC) is a supplement form of cysteine which acts as an anti-oxidant. It may help improve fertility in men by reducing oxidative stress that damages or kills reproductive cells such as spermatozoa.

L-Acetyl Carnitine

L-carnitine is an amino acid derivative that transports fatty acids into your cells to be processed for energy. L-acetyl-carnitine has an additional acetyl molecule which makes it more easily absorbed in the gut and more readily crosses the blood-brain barrier.

Omega Fatty Acids

The omega fatty acids eicosapentaenoic acid (EPA), and docosahexaenoic acid (DHA) are both commonly found in marine oils.

Alpha-Lipoic Acid

Alpha-lipoic acid is a naturally occurring fatty acid that can be found in many foods such as yeast, spinach, broccoli, potatoes, and organ meats such as liver or kidney. It is known to have antioxidant properties.

Selenium Selenium is a mineral which has antioxidant properties.

Coenzyme Q10

Coenzyme Q10 (CoQ10) is also known as ubiqinone and is a nutrient that occurs naturally in the body. CoQ10 is also in many foods we eat. CoQ10 acts as an antioxidant, which protects cells from damage. The amounts of CoQ10 in found naturally in food is much lower than that found in supplements. Good food sources of CoQ10 include cold water fish, like tuna, salmon, mackerel, and sardines, vegetable oils and meats.

Folic Acid

Folic acid, which is converted into folate by the body, is used as a dietary supplement and in food fortification as it is more stable during processing and storage. Folic acid is a type of B vitamin that is normally found in foods such as dried beans, peas, lentils, oranges, whole-wheat products, liver, asparagus, beets, broccoli, brussels sprouts, and spinach.

Vitamin A

Vitamin A compounds are found in both animal and plant foods and come in two different forms: preformed vitamin A and provitamin A.

Preformed vitamin A is known as the active form of the vitamin, which your body can use just as it is. It's found in animal products including meat, chicken, fish and dairy and includes the compounds retinol, retinal and retinoic acid.

Provitamin A carotenoids—alpha-carotene, beta-carotene and beta-cryptoxanthin—are the inactive form of the vitamin found in plants.

Lycopene

Lycopene is a carotenoid having anti-oxidant properties.

Lycopene may be found in foods such as tomatoes, asparagus and parsley. However, lycopene dietary supplements in oil may be more efficiently absorbed than lycopene from food.

Beta Carotene

Beta carotene is a carotenoid having anti-oxidant properties.

β-carotene is converted into vitamin A, an essential vitamin and may be found in food such as onions, carrots, peas, spinach and squash. It may also be administered as a supplement.

Vitamin C

Vitamin C is an essential water-soluble vitamin which cannot be produced endogenously in the body. It is found in many fruits and vegetables, including oranges, strawberries, kiwi fruit, bell peppers, broccoli, kale, and spinach.

Vitamin D

Vitamin D is involved in the regulation of the minerals, calcium and phosphorus found in the body. It also plays an important role in maintaining proper bone structure. Typical sources of Vitamin D are from sunlight. Vitamin D3 also known as cholecalciferol is the recommended form of Vitamin D supplement.

Myoinositol

Myo-inositol is an important growth-promoting factor of mammalian cells and animals. The role of myo-inositol as a lipotropic factor, it also acts as a co-factor of enzymes and in signal transduction. It is a substance found naturally in cantaloupe, citrus fruit, and many fiber-rich foods such as beans, brown rice, corn, sesame seeds, and wheat bran. It is also sold in supplement form.

Nigella sativa Seed Oil

Nigella sativa seed oil is sometimes known as Black cumin seed oil or Kalonji oil. It is oil extracted from the seeds of Nigella sativa, a plant native to southwest Asia. One of the key components of black seed oil is thymoquinone, a compound with antioxidant properties.

Withania somnifera

Withania somnifera is also known commonly as ashwagandha, Indian ginseng, poison gooseberry, or winter cherry, is a plant in the Solanaceae or nightshade family. It may help to increase testosterone levels and through its antioxidant properties boost sperm quality and fertility in men. Typically, it is administered as a root powder that can be dissolved in milk.

Curcumin

Curcumin is produced by Curcuma longa plants and typically in a bright yellow powder. It is the principal curcuminoid of turmeric (Curcuma longa), a member of the ginger family, Zingiberaceae. It has potent anti-inflammatory and antioxidant properties.

Glutathione

Glutathione is an antioxidant capable of preventing damage to important cellular components caused by reactive oxygen species such as free radicals, peroxides, lipid peroxides, and heavy metals. Glutathione is a substance produced naturally by the liver. It is also found in fruits, vegetables, and meats.

In a preferred embodiment of the invention, the recommendation for supplements is to be administered as separate supplements or in combination selected from the group consisting of:

(i) Vitamin E in the amount of 20 to 100 mg/day for 2 to 12 months

(ii) Zinc in the amount of 66 to 500 mg/day for 3 to 6 months

(iii) N-acetyl cysteine in the amount of at least about 600 mg/day

(iv) L-acetyl carnitine in the amount of 1000 to 3000 mg/day

(v) Omega fatty acids selected from DHA in the amount of 400 to 1120 mg/day and EPA in the amount of 135 to 720 mg/day for 12 to 32 weeks

(vi) Alpha-lipoic acid in the amount of 600 mg/day for at least 12 weeks

(vii) Selenium in the amount of 100 to 200 ug/day

(viii) Coenzyme Q in the amount of 100 to 300 mg/day

(ix) Folic acid in the amount of 200 ug to 5 mg/day for at least 26 weeks

(x) Vitamin A in the amount of 1 to 30 mg/day

(xi) Lycopene in the amount of 6 to 8 mg/day

(xii) Beta carotene in the amount of 18 to 30 mg/day

(xiii) Vitamin C in the amount of 500 to 1000 mg/day for 3 to 12 weeks

(xiv) Vitamin D in the amount of at least 15 ug/day

(xv) Myoinositol in the amount of at least about 4 g/day for 2 to 3 months

(xvi) Nigella sativa seed oil in the amount of at least about 5 ml/day or 5 mg/day

(xvii) Withania somnifera in the amount of at least 5 g/day for at least 3 months

(xviii) Curcumin in the amount of at least about 80 mg/day for at least 10 weeks

(xix) Glutathione in the amount of at least about 600 mg/day

In one embodiment, the recommendation for supplements is to be administered in the combination of Vitamin E in the amount of 20 to 1000 mg/day in combination with Vitamin C in the amount of 10 to 1000 mg/day for 2 to 12 months.

In one embodiment, the recommendation for supplements is to be administered in the combination of zinc in the amount of 66 to 500 mg/day in combination with folic acid in the amount of at least about 5 mg/day.

In one embodiment, the recommendation for supplements is to be administered in the combination of I-acetyl carnitine in the amount of 1000 to 3000 mg/day in combination with:

Vitamin C in the amount of at least about 60 mg/day;

Coenzyme Q10 in the amount of at least about 20 mg/day;

Vitamin E in the amount of at least about 10 mg/day;

Vitamin B9 in the amount of at least about 200 ug/day;

Vitamin B12 in the amount of at least about 1 ug/day;

Zinc in the amount of at least about 10 mg/day; and

Selenium in the amount of at least about 50 ug/day.

In another embodiment of the invention, the recommendation for supplements is to be administered in the combination of omega fatty acids from DHA in the amount of 400 to 1120 mg/day in combination with EPA in the amount of 135 to 720 mg/day.

In another embodiment of the invention, the recommendation for supplements is to be administered in the combination of Vitamin A in the amount of 1 to 30 mg/day in combination with:

Selenium in at least about 100 ug/day;

Vitamin C in at least about 10 mg/day; and

Vitamin D in at least about 15 mg/day.

In further embodiment, the recommendation for supplements is to be administered in the combination of Vitamin D3 in the amount of at least about 300,000 IU in combination with calcium in the amount of at least about 500 mg/day for 150 days.

In several embodiments of the invention, the recommendation system recommends specific supplements combined with food items for individuals with sperm conditions who would like to improve their fertility.

Food Items

In some embodiments of the invention, the recommendation system recommends specific food items for individuals with sperm conditions who would like to improve their fertility.

In some embodiments of the invention, the recommendation system recommends specific food items for individuals with sperm conditions who would like to improve their fertility selected from the group:

Walnuts

Walnuts have higher antioxidant activity than most other nuts as they are rich in vitamin E, melatonin and polyphenols.

Fish

Fish especially cold water fish such as salmon or other cold-water fish contain high levels of omega-3 fatty acids which have a positive influence on sperm morphology.

Organ Meats

Organ meats such as liver, kidney, sweet breads, stomach, tongue, heart, brain serving at least 1 serving per week was associated with higher semen quality. This is due to increased copper intake as well as high levels of Vitamins A and B and Coenzyme Q10.

Low Fat Dairy Foods

Low fat dairy food such as low fat milk, yogurt, and cottage cheese are associated with higher percent progressively motile sperm. The potential mechanism of action may be due to effects on circulating insulin growth factor-1 (IGF-1) and insulin levels which are important for sperm formation.

Vegetable and Fruits

Adequate fruit and vegetable consumption may contribute to sperm health by acting as anti-oxidants.

Per 2000 kcal/day diet one should consume at least 700 g total vegetables and fruits. Of the at least 400 g vegetables at least 100 g are dark, leafy vegetables. Of the at least 300 g fruit, at least 20 g are tomatoes.

In a preferred embodiment of the invention, the recommendation for food is to be selected from the group consisting of:

(i) walnuts in the amount of at least 75 g/day;

(ii) fish in the amount of at least 1.5 to 2.4 servings per week;

(iii) organ meat in the amount of at least one serving per week;

(iv) low fat dairy foods in the amount of at least 1 to 4 servings per week; and

(v) vegetables and fruits in the amount of at least 5 to 7 servings per day.

In one preferred embodiment, the low fat dairy food comprises low fat milk in the amount of at least 1 serving per day.

Specific Diet and Lifestyle Changes

In some embodiments of the invention, the recommendation system recommends specific diets or lifestyle changes for individuals with sperm conditions who would like to improve their fertility.

In one embodiment of the invention, the recommendation system recommends avoidance of processed meats.

In one embodiment of the invention, the recommendation system recommends avoidance of full fat dairy products and cheese.

In one embodiment of the invention, the recommendation system recommends avoidance of sweets.

In one embodiment of the invention, the recommendation system recommends avoidance of trans-fats and saturated fats.

In one embodiment of the invention, the recommendation system recommends caloric restriction with and without high protein in obese men with a BMI over 25 kg/m2, more preferably with a BMI over 30 kg/m2 to order to lose weight.

In one embodiment of the invention, the recommendation system recommends reducing alcoholic beverages and sugar sweetened beverages to less than one per day.

In one embodiment of the invention, the recommendation system recommends consuming soy-based products in moderation.

In several embodiments of the invention, the dietary recommendations of the present invention may contribute to improve sperm related conditions.

In a preferred embodiment of the invention, the sperm related-condition is low sperm count below 15 million sperm per milliliter.

In a preferred embodiment of the invention, the sperm-related condition is low sperm motility with less than 40% sperm motility or less than 32% with progressive sperm motility.

In a preferred embodiment of the invention, the sperm-related condition is abnormal sperm morphology with less than 30% morphologically normal sperm.

In a preferred embodiment of the invention, the sperm-related condition is low sperm concentration with less than 20×106/mL concentration in semen.

In a preferred embodiment of the invention, the sperm-related condition is associated with low testosterone below 270 to 1,070 nanograms per deciliter (ng/dL).

In another preferred embodiment, the sperm-related condition is associated with an individual who is obese and may require a restricted caloric intact to lose weight. For example, an individual with sperm-related conditions associated with a BMI 30.0 or greater.

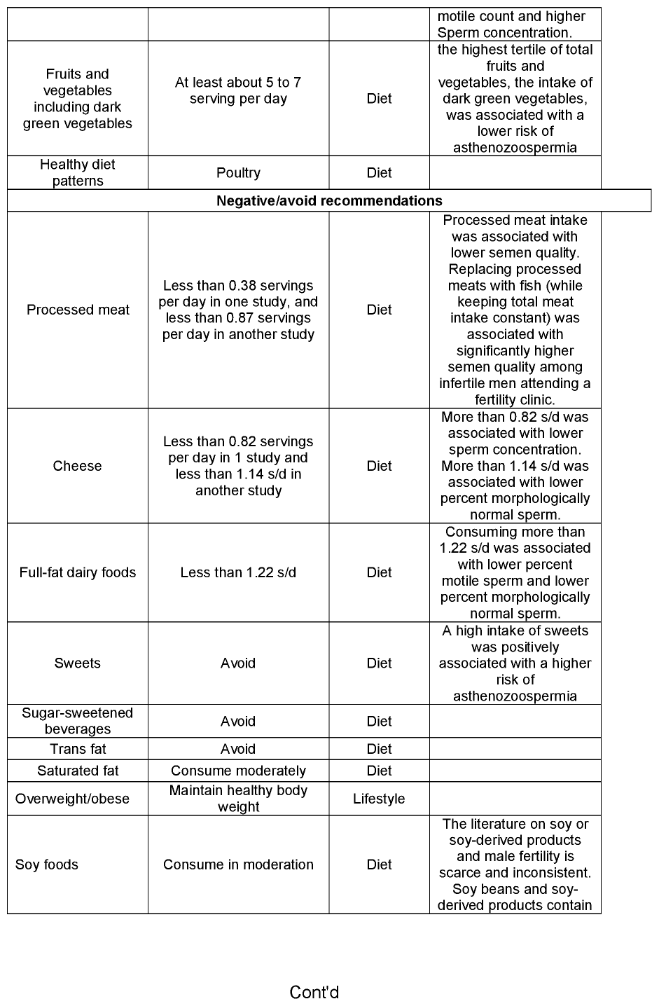

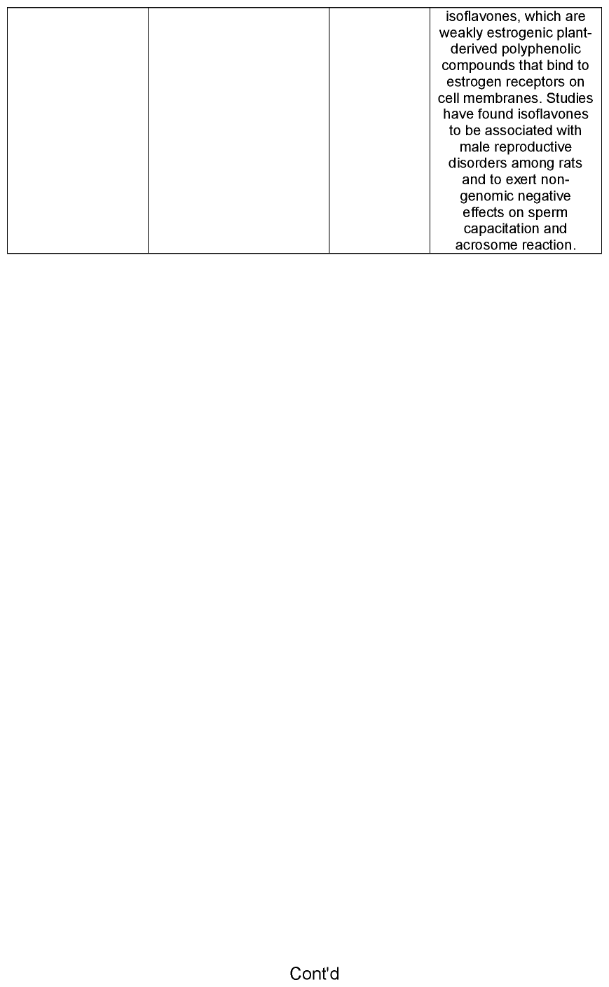

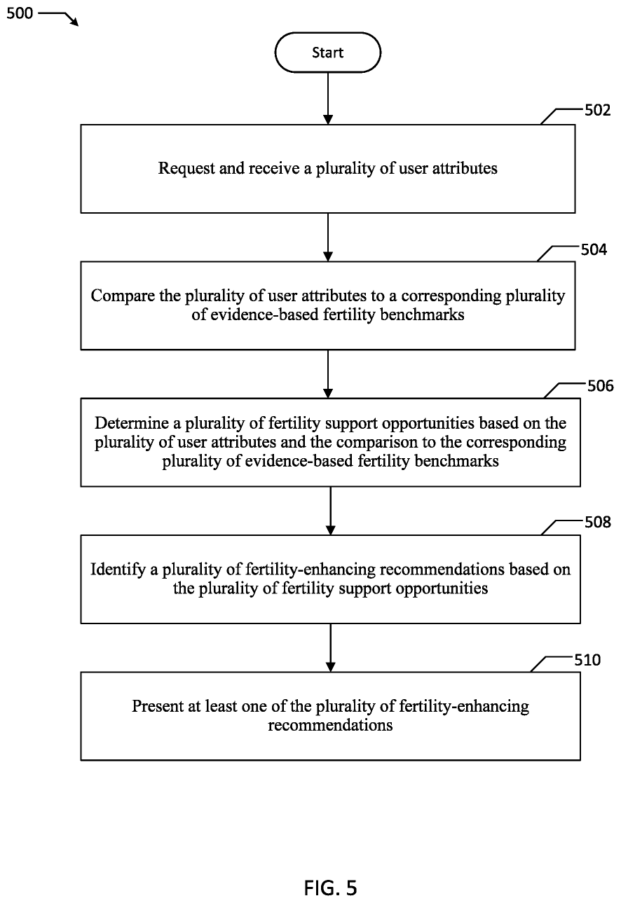

FIG. 5 illustrates an example embodiment of a method 500 of the presently disclosed method, as was discussed above in relation to the system 100. The method 500 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 500 may also be implemented by a set of instructions stored on a computer readable medium that, when executed by a processor, cause the computer system to perform the method. For example, all or part of the method 500 may be implemented by the CPU 120 and memory 118. Although the examples below are described with reference to the flowchart illustrated in FIG. 5, many other methods of performing the acts associated with FIG. 5 may be used. For example, the order of some of the blocks may be changed, certain blocks may be combined with other blocks, one or more of the blocks may be repeated, and some of the blocks described may be optional.

Block 502 can include requesting and receiving a plurality of user attributes 122. For example, a display 106 may present an attribute questionnaire 124 to solicit answers 126, to which the user device 102 provides the answers 126 to then be selected as user attributes 122. In block 504, comparisons of the plurality of user attributes 122 to a corresponding plurality of evidence-based fertility benchmarks 128 may occur. Based on these comparisons, at block 506, a plurality of fertility support opportunities 138 can be determined based on the plurality of user attributes 122 and the comparison to the corresponding plurality of evidence-based fertility benchmarks 128. At block 508, an embodiment of method 500 may identify a plurality of fertility enhancing recommendations 140 based on the plurality of fertility support opportunities 138. For example, the evidence-based diet and lifestyle recommendation engine 112 may comprise a cloud-based system trained to interpret fertility support opportunities to provide recommendations 140. Lastly, at block 510, at least one of the plurality of fertility enhancing recommendations 140 can be presented.

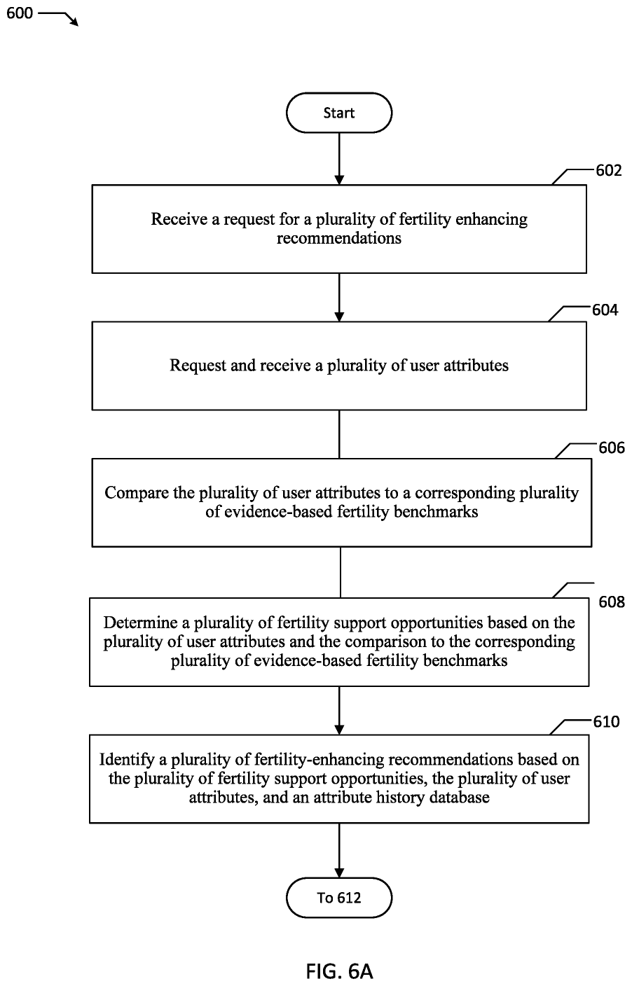

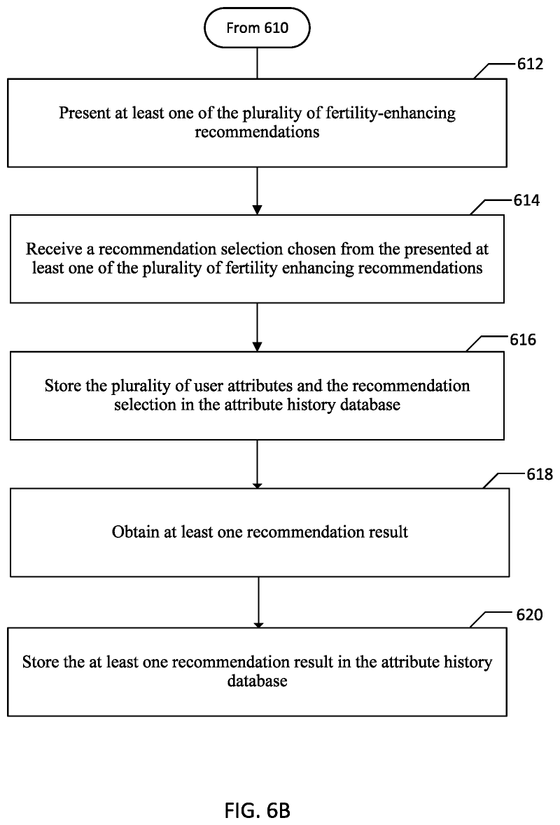

FIGS. 6A and 6B disclose an exemplary embodiment of a method 600 of the presently disclosed method. The method 600 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 600 may also be implemented by a set of instructions stored on a computer readable medium that, when executed by a processor, cause the computer system to perform the method. For example, all or part of the method 600 may be implemented by the CPU 120 and memory 118. Although the examples below are described with reference to the flowchart illustrated in FIG. 6, many other methods of performing the acts associated with FIG. 6 may be used. For example, the order of some of the blocks may be changed, certain blocks may be combined with other blocks, one or more of the blocks may be repeated, and some of the blocks described may be optional.

Block 602 may include receiving a request for a plurality of fertility enhancing recommendations 140. For example, a user may submit a request for the fertility enhancing recommendations 140 through any number of methods, including: opening an application on the user device 102, making a formal request through an application on the user device 102, submitting a request for periodic fertility enhancing recommendations 140 through the user device 102, signing into an online account through a web browser, making a formal request through a web browser, or submitting a request for periodic fertility enhancing recommendations 140 through the web browser.

At block 604, the recommendation system 104 may request and receive a plurality of user attributes 122. For example, the recommendation system 104 may present an attribute questionnaire 124 to the user. This attribute questionnaire 124 may be a standard questionnaire or a questionnaire that is customized based on known preliminary attributes, or answers to prior questions. In another example, the recommendation system 104 may request the plurality of user attributes 122 by providing a list of available home test kits, that a user may use at home. Then, after the test has been performed, the recommendation system 104 may receive the results from the test and, based on these results, determine the user attributes 122 related to such a test. For example, the home test kit may be an application to track the timing of the ovulation cycle of the user to determine the best dates for conception which may be monitored by a further application on an additional user device.

In another example, at block 604, the recommendation system 104 may provide a self-assessment tool. Similar to the prior example, the user will may make use of this self-assessment tool, submitting the results to the recommendation system 104. Again, based on the received results, the recommendation system 104 may determine the user attributes 122 based on the test. In yet another example, the recommendation system 104 may request the user have a standardized health test performed by a medical professional. In this example, the results of this performed health test may be submitted to the recommendation system 104, which thereby determines the user attributes 122 based on the results. Though some specific examples as to external tests have been given, these examples are non-limiting as the recommendation system 104 may be configured to receive results of any external or third party test in order to determine the corresponding user attributes 122.

In Block 606, the recommendation system 104 may be configured to compare the plurality of user attributes 122 to a corresponding plurality of evidence-based fertility benchmarks 128. For example, these evidence-based fertility benchmarks 128 may include standardized benchmarks, as in benchmarks that are given to all, regardless of individual variances. In another example, these benchmarks 128 may be customized based on a particular user's history or goals. For example, if a healthy user is trying to improve his or her fertility and the current user attributes 122 exceeds all standard evidence-based fertility benchmarks 128, the recommendation system 104 may be configured to determine a customized fertility benchmark set 132 for which the particular user should aim. In contrast, in another example, a different user that is far below a standard evidence-based fertility benchmark 128 may be compared to a different, lower benchmark value as a manner of inspiring progress and providing milestones.

The example method, at Block 608, may be configured to determine a plurality of fertility support opportunities 138 based on the plurality of user attributes 122 and the comparison to the corresponding plurality of evidence-based fertility benchmarks 128. For example, the recommendation system 104 may determine that a user attribute 122 corresponds to an above optimal stress level. Based on this comparison, the recommendation system 104 may determine a fertility support opportunity 138 to reduce stress. In another example, the recommendation system 104 may determine that the user has not yet seen a medical professional, and, as such, determine a fertility support opportunity 138 to visit a medical professional.

At Block 610, the recommendation system 104 may identify a plurality of fertility-enhancing recommendations 140 based on at least the plurality of fertility support opportunities 138. For example, the recommendation system may determine a plurality of similar prior cases by analyzing the attribute history database 148, identifying similarities between the user attributes 122 received and the plurality of prior user attributes within the attribute history database 148. For example, the recommendation system 104 may identify that the user attributes 122 detail a user with an above average BMI and other similarities that correspond to a particular group of past users and therefore the cases of those member of that particular group of past users are determined as similar prior cases.

Furthermore, in this example, the recommendation system 104 may determine a plurality of prior case results based on the plurality of similar prior cases. As detailed previously, the attribute history database 148 may comprise corresponding recommendations associated with prior user attributes, and the effectiveness of these corresponding recommendations. As such, the recommendation system 104 may analyze the corresponding recommendations and their effectiveness as associated with the particular group of past users to determine a plurality of prior case results.

Furthermore, in this example, the recommendation system 104 may determine successful recommendations and a plurality of unsuccessful recommendations based on a plurality of prior case results. For example, the recommendation system 104 may have recommended the users in that particular group of past users increase exercise levels in some cases and decrease food consumption in other cases. Based on prior case results as determined based on the attribute history database 148, the recommendation system 104 may determine that the recommendations for decreasing food consumption were not very successful, yet increasing exercise levels proved to be very successful and, as such, determines that increasing exercise levels is a successful recommendation whereas decreasing food consumption is an unsuccessful recommendation. By conducting analysis of these prior user attributes, the recommendation selection and the effectiveness of corresponding recommendations, the recommendation system 104 may identify trends associated with different subset patient populations, thereby creating and validating a plurality of lifestyle interventions. These examples of successful and unsuccessful recommendations are non-limiting, as different groups may experience different levels of success to the same recommendations.

In addition, the recommendation system 104 may be configured to determine a plurality of fertility enhancing recommendations based on the plurality of successful recommendations and the plurality of unsuccessful recommendations. For example, the recommendation system 104 may be configured to only recommend the plurality of successful recommendations. In another example, the recommendation system 104 may still recommend any of the unsuccessful recommendations. The recommendation system 104 may make these recommendations based on any number of reasons, including a slight difference in the user attributes 122 as compared to the prior user attributes, a lack of insufficient data to support a true unsuccessful recommendation, or data supporting that, although unsuccessful, the recommendation is popular and often followed through by users. In another example, the recommendation system 104 may recommend less than all of the plurality of successful recommendations. In an example, the decision for selecting which of the plurality of recommendations to present generate may be performed by an AI.

In another example, successful recommendations may be based on guidelines associated with particular medical conditions, such as a user with sperm conditions such as low sperm count. In that case, these guidelines would be determined as successful recommendations.

At Block 612, the recommendation system may present at least one of the plurality of fertility-enhancing recommendations 140. At Block 614, the recommendation system 104 may receive a recommendation selection chosen from the presented at least one of the plurality of fertility enhancing recommendations 140. For example, a user may be presented with three fertility-enhancing recommendations 140, to follow avoid consumption of processed meats; to increase consumption of fruits and vegetables; and to reduce alcohol consumption to less than one drink per day. The user may select one, two, or all three of these options. As such, the recommendation system 104 receives, from the user device 102, these two selected recommendations as the recommendation selection. In another example, the user may not select any of the presented recommendations, at which point the recommendation system 104 may generate and present a different plurality of fertility enhancing recommendations 140.

In another example, after the user reviews the presented fertility enhancing recommendations 140, the user may submit a request to contact a fertility coach. For example, the user may be undecided as to how to implement the recommendations or may simply have a question for which the user seeks an answer. In some instances, the recommendation system 104 may determine that the question for which the user seeks an answer may be adequately answered by a virtual coach, and thereby provides access to and interaction with said virtual coach. In other instances, the recommendation system 104 may determine that the question will be best handled by a personal coach, a living individual, and thereby provides access to and interaction with said personal coach.

At Block 616, the recommendation system 104 may store the plurality of user attributes 122 and the recommendation selection in the attribute history database 148. For example, the recommendation system 104 may store all user attributes 122 received on a first day, along with the recommendation selection received on that same first day. These user attributes 122 and recommendation selection may then be accessed by the recommendation system 104 in the future when analyzing the attribute history database 148.

The recommendation system 104 may obtain at least one recommendation result at Block 618. In an example, the user may submit a recommendation result through the user device 102. This result may include a qualitative or quantitative rating as selected by the user. In another example, the recommendation system 104 may receive a future plurality of user attributes 122 and, at that time, compare the received future user attributes with the previously received user attributes, now prior user attributes, within the attribute history database 148. Based on this comparison, the recommendation system 104 may determine a recommendation result, such as decreased or increased BMI. After obtaining this recommendation result, the recommendation system 104 may store the at least one recommendation result in the attribute history database 148, corresponding to the prior recommendation selection. This recommendation system 104 may then await another request for fertility enhancing recommendations 140, and at that time, perform the method 600 again at Block 602.

Such an example method as disclosed in FIGS. 6A and 6B allow for the continuous, customized, integrated recommendation system 104 to endlessly improve upon recommendations as the attribute history database 148 grows in size. Through this growth, the recommendation system 104, and in some embodiments, the evidence-based diet and lifestyle recommendation engine 112, will have an ever-expanding set of data from which it can derive fertility enhancing recommendations 140, with increasing particularity regarding what users receive which recommendations.

In another aspect, a method of treatment may comprise using any of the above described systems or methods to generate any one or more of the fertility enhancing recommendations 140, diet and lifestyle recommendations, or specific supplementation recommendations. Furthermore, the method of treatment may comprise administering a treatment based on at least the any of one or more of the fertility enhancing recommendations 140, diet and lifestyle recommendations, or specific supplementation recommendations to a user. For example, when the recommendation system 104 determines a fertility enhancing recommendation 140 that comprises increasing a user's zinc to 500 mg/day by way of a supplement, an example method of treatment may comprise administering a treatment comprising a zinc supplement of 500 mg to the user each day.

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1: Dietary Recommendations for Individuals with Sperm Conditions

The following are dietary recommendations or supplements for individual with sperm-related conditions.

TABLE 1
Dietary Recommendations

| | | | | |
|---|---|---|---|---|
| | | Supplement | | |
| Diet and lifestyle | Dietary | or Diet or | | |
| components | recommendation | lifestyle | Tips for the consumer | Ref |

Positive recommendations

| | | | | |
|---|---|---|---|---|
| Vitamin E | 20-1000 mg/day for 2-12 | Supplement | In RCTs, vitamin E has | 14, 16, |
| | months | | been shown to increase | 27-29 |
| | either alone or in | | pregnancy rate in the | |
| | combination with | | female partner and | |
| | 10 to 1000 mg/day | | semen quality. Vitamin | |
| | vitamin C | | E neutralises free | |
| | | | radicals and protects | |
| | | | cellular membrane | |
| | | | against oxygen free | |
| | | | radicals. Of note, the | |
| | | | Tolerable Upper Intake | |
| | | | Level of vitamin E is | |
| | | | 1000 mg/d. | |
| Zinc | 66 to 500 mg/day zinc for | Supplement | In RCTs, zinc has been | 16, 43- |
| | 3-6 months | | shown to increase | 45 |
| | Alone or in combination | | pregnancy rate in the | |
| | with at least about 5 | | female partner and | |
| | mg/day folic acid | | semen quality. Zinc | |
| | | | plays crucial roles in | |
| | | | male reproduction such | |
| | | | as DNA replication, | |
| | | | protects against reactive | |
| | | | oxygen species etc. | |
| N-acetylcysteine | At least about 600 mg/day | Supplement | Scavenges reactive | 14, 16, |
| (NAC) | | | oxygen species | 30-32 |
| L-acetyl carnitine | 1000-3000 mg/day for 6 | Supplements | can significantly improve | 14, 16, |
| (LAC) | months alone or in | | both sperm | 30, 33 |
| | combination (below) | | concentration and total | |
| | | | sperm counts among | |
| | | | men with astheno- or | |
| | | | oligoasthenozoospermia. | |
| | | | help transport PUFA to | |
| | | | mitochondria and have | |
| | | | antioxidant properties. | |
| | | | Neutralizes free radicals | |
| | | | and acts as an energy | |
| | | | source. | |
| L-carnitine | 2000 mg/day for 2-3 | Supplements | can significantly improve | 14, 16, |
| | months alone or in | | both sperm | 30, 33- |
| | combination with: | | concentration and total | 36 |
| | L-Carnitine (1500 mg), | | sperm counts among | |
| | vitamin C (60 mg), | | men with astheno- or | |
| | coenzyme Q10 (20 mg), | | oligoasthenozoospermia. | |
| | vitamin E (10 mg), vitamin | | help transport PUFA to | |
| | B9 (200 μg), vitamin B12 | | mitochondria and have | |
| | (1 μg), Zinc (10 mg) and | | antioxidant properties. | |
| | selenium (50 μg). | | Neutralizes free radicals | |
| | | | and acts as an energy | |
| | | | source. | |
| Omega fatty acids: | DHA 400-800 mg/d alone | Supplements | PUFAs are used in the | 16, 42 |
| EPA and DHA | or in combination with | | cell membrane of | |
| | EPA 135 mg/d for 3 | | spermatozoa. | |
| | months | | Successful fertilization | |
| | EPA 0.72 g/day and DHA | | of spermatozoa is | |
| | 1.12 g/day for 32 weeks | | dependent on the lipid | |
| | | | composition of the | |
| | | | membrane. | |
| Alpha-lipoic acid | 600 mg/day for 12 weeks | Supplements | Exogenous | 4, 5 |
| (ALA) | | | ALA supplementation | |
| | | | results in increased | |
| | | | unbound ALA | |
| | | | levels, which can act as | |
| | | | a strong antioxidant and | |
| | | | improve oxidative stress | |
| | | | status both in vitro and | |
| | | | in vivo. | |
| Selenium | 100-200 μg/day for 3-6.5 | Supplements | increase in total sperm | 46-48 |
| | months | | motility compared with | |
| | | | placebo. Enhancement | |
| | | | of enzymatic antioxidant | |
| | | | activity. Required for | |
| | | | normal male | |
| | | | reproductive function, | |
| | | | testicular development, | |
| | | | spermatogenesis, and | |
| | | | spermatozoa motility | |
| | | | and function. Necessary | |
| | | | for the reduction of | |
| | | | antioxidant enzymes. | |
| | | | Protects against | |
| | | | oxidative DNA damage. | |
| Arginine | | Supplements | evidence not sufficient | |
| | | | to make a | |
| | | | recommendation on | |
| | | | dose or duration of | |
| | | | treatment | |
| CoQ10 (co-enzyme | 100-300 mg/day | Supplements | Scavenges free radicals | 14, 37- |
| Q10 or Ubiquinol) | | | | 41 |
| Folic acid | 200 ug to 5 mg/day for 26 | Supplements | Scavenges free radicals | 17 |
| | weeks | | | |
| Vitamin A | 1-30 mg/day alone or in | Supplements | Scavenges free radicals | EFSA, |
| | combination with | | Men with lower serum | Dietary |
| | selenium (100 μg/day) | | concentration of retinol | Reference |
| | and vitamin C (10 | | have worse sperm | Values |
| | mg/day) and vitamin D | | quality. Combinations of | (DRV) |
| | (15 mg/day) | | antioxidants with vitamin | 2017 |
| | | | A in the range of 1-30 | |
| | | | mg/d may be beneficial | |
| Lycopene | 6-8 mg/day | Supplements | Improve sperm | 12-25 |
| | | | parameters | |
| Beta carotene | 18-30 mg/day | Supplements | Lycopene is detected at | 12-15 |
| | | | high concentrations | |
| | | | in human testes and | |
| | | | seminal plasma with | |
| | | | levels that tend to be | |
| | | | lower in infertile men | |
| Vitamin C | 500-1000 mg/day for 3 | Supplements | increase in progressive | 14, 20- |
| | weeks to 3 months | | sperm motility. Levels | 23 |
| | | | of ascorbic acid are 10- | |
| | | | times higher in seminal | |
| | | | plasma than in blood | |
| | | | plasma. Ascorbic acid | |
| | | | diminishes DNA | |
| | | | damage by scavenging | |
| | | | free radicals. | |
| Vitamin D | 15 ug/d (avoiding | Supplements | Low vitamin D status | 24, 25 |
| | deficiency) or | | has been associated | |
| | supplementation if | | with low semen quality. | |
| | deficient | | Infertile men with | |
| | Cholecalciferol 300,000 | | vitamin D deficiency | |
| | IU initially, then 1400 IU | | have significantly lower | |
| | cholecalciferol and 500 | | sperm production and | |
| | mg of calcium daily for | | motility compared with | |
| | 150 days | | men having normal | |
| | | | vitamin D status. The | |
| | | | vitamin D receptor is | |
| | | | present in human testis, | |
| | | | epididymis, seminal | |
| | | | vesicles, and | |
| | | | spermatozoa and | |
| | | | induces non-genomic | |
| | | | effects on | |
| | | | spermatogenesis. | |
| | | | Vitamin D promotes | |
| | | | intracellular calcium | |
| | | | uptake. A higher live | |
| | | | birth rate was observed | |
| | | | among oligozoospermic | |
| | | | men in the high-dose | |
| | | | vitamin D | |
| | | | supplementation | |
| | | | compared to placebo. | |
| Walnuts | At least about 75 g/day | Diet | PUFAs are used in the | 10, 11 |
| | | | cell membrane of | |
| | | | spermatozoa, | |
| | | | successful fertilization of | |
| | | | spermatozoa is | |
| | | | dependent on the lipid | |
| | | | composition of the | |
| | | | membrane. | |
| Myoinositol | At least about 4 g/day for | Supplements | increase in sperm | 14, 23, |
| | 2-3 months | | concentration, | 26 |
| | | | progressive motility and | |
| | | | count | |
| Nigella sativa L. | At least about 5 ml/day or | Supplement | increase in sperm count, | 3 |
| seed oil | 5 mg/day for 2 months | | motility and morphology | |
| | | | compared to placebo | |
| Withania somnifera | root powder | Supplements | Semen parameters | 6-8 |
| (also known as | (at least about 5 g/day) | | improved post- | |
| Indian ginseng) | orally for 3 months with | | treatment. Treatment | |
| | milk | | with W. somnifera | |
| | | | significantly | |
| | | | improved the activity of | |
| | | | superoxide | |
| | | | dismutase and catalase | |
| | | | and the level of | |
| | | | glutathione, and | |
| | | | reduced the levels of | |
| | | | lipid peroxides and | |
| | | | protein carbonyl groups | |
| | | | in infertile men. | |
| | | | Treatment with W. | |
| | | | somnifera improved | |
| | | | levels of vitamins | |
| | | | A, C, and E, indicating | |
| | | | the strength of this herb | |
| | | | to protect against | |
| | | | infertility due to vitamin | |
| | | | loss. | |
| Curcumin | At least about 80 mg/day | Supplements | Curcumin improved | 9 |
| | for 10 weeks | | semena quality | |
| | | | parameters among | |
| | | | oligoasthenoteratozoospermic | |
| | | | men. | |
| | | | Curcumin has an | |
| | | | antioxidative effect by | |
| | | | down-regulating the | |
| | | | levels of inflammatory | |
| | | | mediators, such as | |
| | | | cyclooxygenase (COX)- | |
| | | | 1, CRP, TNF-α, IFN-γ, | |
| | | | and nuclear transcription | |
| | | | factor (NF)-κB. | |
| Glutathione | At least about 600 mg/day | Supplements | Improves sperm | 6-8 |
| | | | concentration, motility, | |
| | | | and morphology | |

Negative/avoid recommendations

| | | | | |
|---|---|---|---|---|
| Alcohol | Consume moderately | diet | Excessive alcohol | 68, 69 |
| | | | decreases hormones in | |
| | | | men and degenerates | |
| | | | sperm cells. The | |
| | | | negative impact of | |
| | | | alcohol on semen | |
| | | | parameters is dose | |
| | | | dependent; only at very | |
| | | | high quantities and | |
| | | | among alcoholics. | |

Non randomized controlled trials:

| | | | | |
|---|---|---|---|---|
| | | Supplements | | |
| Diet and lifestyle | Dietary | or Diet or | | |
| components | recommendation | lifestyle | Tips for the consumer | Ref |

Positive recommendations

| | | | | |
|---|---|---|---|---|
| Fish | More than 1.5 servings | Diet | Among infertile men | 49- |
| | per week, preferably | | attending a fertility | 51 |
| | greater than 2.4 servings | | center, consuming more | |
| | per week | | than 1.5 servings of fish | |
| | | | per week was associated | |
| | | | with a higher percent of | |
| | | | morphologically normal | |
| | | | sperm and more than | |
| | | | 2.4 servings per week | |
| | | | with higher sperm count. | |
| Organ meat | Recommend liver, kidney, | Diet | Consuming any amount | 49 |
| | sweet breads, stomach, | | of organ meat was | |
| | tongue, heart, brain | | associated with higher | |
| | serving at least 1 serving | | semen quality. In one | |
| | per week | | study, this association | |
| | | | was explained by | |
| | | | copper intake. High | |
| | | | levels of Vitamins A and | |
| | | | B as well as Coenzyme | |
| | | | Q10. | |
| Low-fat dairy foods | At least about 1 to 4 | Diet | Consuming more than | 50- |
| (Includes low fat | servings per day | | 0.77 s/d was associated | 52 |
| milk, yogurt, and | | | with higher percent | |
| cottage cheese) | | | progressively motile | |
| | | | sperm. | |
| | | | Potential mechanistic | |
| | | | pathway: this finding | |
| | | | may reflect known | |
| | | | effects of low-fat dairy | |
| | | | intake on circulating | |
| | | | insulin growth factor-1 | |
| | | | (IGF-1) and insulin | |
| | | | levels. Given that | |
| | | | spermatogenesis is a | |
| | | | process of active cell | |
| | | | division requiring insulin | |
| | | | and that IGF-1 can bind | |
| | | | and activate Leydig cell | |
| | | | insulin receptors. | |
| Low-fat milk | At least about 1 serving | Diet | More than 0.14 s/d for | 50- |
| (Includes skim milk | per day | | progressive motility and | 52 |
| and 1% and 2% | | | more than 0.80 s/d for | |
| milk) | | | Sperm Concentration- | |
| | | | Associated with higher | |
| | | | percent progressively | |
| | | | motile sperm and total | |
| | | | motile count and higher | |
| | | | Sperm concentration. | |
| Fruits and | At least about 5 to 7 | Diet | the highest tertile of total | 50, |
| vegetables | serving per day | | fruits and vegetables, the | 51, |
| including dark | | | intake of dark green | 54 |
| green vegetables | | | vegetables, was associated | |
| | | | with a lower risk of | |
| | | | asthenozoospermia | |
| Healthy diet | Poultry | Diet | | 51, |
| patterns | | | | 55- |
| | | | | 59 |

Negative/avoid recommendations

| | | | | |
|---|---|---|---|---|
| Processed meat | Less than 0.38 servings | Diet | Processed meat intake | 50, |
| | per day in one study, and | | was associated with | 57, |
| | less than 0.87 servings | | lower semen quality. | 59, |
| | per day in another study | | Replacing processed | 66- |
| | | | meats with fish (while | 68 |
| | | | keeping total meat | |
| | | | intake constant) was | |
| | | | associated with | |
| | | | significantly higher | |
| | | | semen quality among | |
| | | | infertile men attending a | |
| | | | fertility clinic. | |
| Cheese | Less than 0.82 servings | Diet | More than 0.82 s/d was | 50, |
| | per day in 1 study and | | associated with lower | 52 |
| | less than 1.14 s/d in | | sperm concentration. | |
| | another study | | More than 1.14 s/d was | |
| | | | associated with lower | |
| | | | percent morphologically | |
| | | | normal sperm. | |
| Full-fat dairy foods | Less than 1.22 s/d | Diet | Consuming more than | 50- |
| | | | 1.22 s/d was associated | 52 |
| | | | with lower percent | |
| | | | motile sperm and lower | |
| | | | percent morphologically | |
| | | | normal sperm. | |
| Sweets | Avoid | Diet | A high intake of sweets | 51 |
| | | | was positively associated | |
| | | | with a higher risk of | |
| | | | asthenozoospermia | |
| Sugar-sweetened | Avoid | Diet | | 50, |
| beverages | | | | 57, |
| | | | | 59, |
| | | | | 66- |
| | | | | 68 |
| Trans fat | Avoid | Diet | | 50, |
| | | | | 57, |
| | | | | 59, |
| | | | | 66- |
| | | | | 68 |
| Saturated fat | Consume moderately | Diet | | 50, |
| | | | | 57, |
| | | | | 59, |
| | | | | 66- |
| | | | | 68 |
| Overweight/obese | Maintain healthy body | Lifestyle | | 54- |
| | weight | | | 70 |
| Soy foods | Consume in moderation | Diet | The literature on soy or | 62, |
| | | | soy-derived products | 63 |
| | | | and male fertility is | |
| | | | scarce and inconsistent. | |
| | | | Soy beans and soy- | |
| | | | derived products contain | |
| | | | isoflavones, which are | |
| | | | weakly estrogenic plant- | |
| | | | derived polyphenolic | |
| | | | compounds that bind to | |
| | | | estrogen receptors on | |
| | | | cell membranes. Studies | |
| | | | have found isoflavones | |
| | | | to be associated with | |
| | | | male reproductive | |
| | | | disorders among rats | |
| | | | and to exert non- | |
| | | | genomic negative | |
| | | | effects on sperm | |
| | | | capacitation and | |
| | | | acrosome reaction. | |

| | | | | |
|---|---|---|---|---|
| | | Supplements | | |
| Diet and lifestyle | Dietary | or Diet or | | |
| components | recommendation | lifestyle | Tips for the consumer | Ref |

Positive recommendations

| | | | | |
|---|---|---|---|---|
| Fish | More than 1.5 servings | Diet | Among infertile men | 49- |
| | per week, preferably | | attending a fertility | 51 |
| | greater than 2.4 servings | | center, consuming more | |
| | per week | | than 1.5 servings of fish | |
| | | | per week was | |
| | | | associated with a higher | |
| | | | percent of | |
| | | | morphologically normal | |
| | | | sperm and more than | |
| | | | 2.4 servings per week | |
| | | | with higher sperm count. | |
| Organ meat | Recommend liver, kidney, | Diet | Consuming any amount | 49 |
| | sweet breads, stomach, | | of organ meat was | |
| | tongue, heart, brain | | associated with higher | |
| | serving at least 1 serving | | semen quality. In one | |
| | per week | | study, this association | |
| | | | was explained by | |
| | | | copper intake. High | |
| | | | levels of Vitamins A and | |
| | | | B as well as Coenzyme | |
| | | | Q10. | |
| Low-fat dairy foods | At least about 1 to 4 | Diet | Consuming more than | 50- |
| (Includes low fat | servings per day | | 0.77 s/d was associated | 52 |
| milk, yogurt, and | | | with higher percent | |
| cottage cheese) | | | progressively motile | |
| | | | sperm. | |
| | | | Potential mechanistic | |
| | | | pathway: this finding | |
| | | | may reflect known | |
| | | | effects of low-fat dairy | |
| | | | intake on circulating | |
| | | | insulin growth factor-1 | |
| | | | (IGF-1) and insulin | |
| | | | levels. Given that | |
| | | | spermatogenesis is a | |
| | | | process of active cell | |
| | | | division requiring insulin | |
| | | | and that IGF-1 can bind | |
| | | | and activate Leydig cell | |
| | | | insulin receptors. | |
| Low-fat milk | At least about 1 serving | Diet | More than 0.14 s/d for | 50- |
| (Includes skim milk | per day | | progressive motility and | 52 |
| and 1% and 2% | | | more than 0.80 s/d for | |
| milk) | | | Sperm concentration- | |
| | | | Associated with higher | |
| | | | percent progressively | |
| | | | motile sperm and total | |
| | | | motile count and higher | |
| | | | Sperm concentration. | |
| Fruits and | At least about 5 to 7 | Diet | the highest tertile of total | 50, |
| vegetables | serving per day | | fruits and | 51, |
| including dark | | | vegetables, the intake of | 54 |
| green vegetables | | | dark green vegetables, | |
| | | | was associated with a | |
| | | | lower risk of | |
| | | | asthenozoospermia | |
| Healthy diet | Poultry | Diet | | 51, |
| patterns | | | | 55- |
| | | | | 59 |

Negative/avoid recommendations

| | | | | |
|---|---|---|---|---|
| Processed meat | Less than 0.38 servings | Diet | Processed meat intake | 50, |
| | per day in one study, and | | was associated with | 57, |
| | less than 0.87 servings | | lower semen quality. | 59, |
| | per day in another study | | Replacing processed | 66- |
| | | | meats with fish (while | 68 |
| | | | keeping total meat | |
| | | | intake constant) was | |
| | | | associated with | |
| | | | significantly higher | |
| | | | semen quality among | |
| | | | infertile men attending a | |
| | | | fertility clinic. | |
| Cheese | Less than 0.82 servings | Diet | More than 0.82 s/d was | 50, |
| | per day in 1 study and | | associated with lower | 52 |
| | less than 1.14 s/d in | | sperm concentration. | |
| | another study | | More than 1.14 s/d was | |
| | | | associated with lower | |
| | | | percent morphologically | |
| | | | normal sperm. | |
| Full-fat dairy foods | Less than 1.22 s/d | Diet | Consuming more than | 50- |
| | | | 1.22 s/d was associated | 52 |
| | | | with lower percent | |
| | | | motile sperm and lower | |
| | | | percent morphologically | |
| | | | normal sperm. | |
| Sweets | Avoid | Diet | A high intake of sweets | 51 |
| | | | was positively | |
| | | | associated with a higher | |
| | | | risk of | |
| | | | asthenozoospermia | |
| Sugar-sweetened | Avoid | Diet | | 50, |
| beverages | | | | 57, |
| | | | | 59, |
| | | | | 66- |
| | | | | 68 |
| Trans fat | Avoid | Diet | | 50, |
| | | | | 57, |
| | | | | 59, |
| | | | | 66- |
| | | | | 68 |
| Saturated fat | Consume moderately | Diet | | 50, |
| | | | | 57, |
| | | | | 59, |
| | | | | 66- |
| | | | | 68 |
| Overweight/obese | Maintain healthy body | Lifestyle | | 54- |
| | weight | | | 70 |
| Soy foods | Consume in moderation | Diet | The literature on soy or | 62, |
| | | | soy-derived products | 63 |
| | | | and male fertility is | |
| | | | scarce and inconsistent. | |
| | | | Soy beans and soy- | |
| | | | derived products contain | |
| | | | isoflavones, which are | |
| | | | weakly estrogenic plant- | |
| | | | derived polyphenolic | |
| | | | compounds that bind to | |
| | | | estrogen receptors on | |
| | | | cell membranes. Studies | |
| | | | have found isoflavones | |
| | | | to be associated with | |
| | | | male reproductive | |
| | | | disorders among rats | |
| | | | and to exert non- | |
| | | | genomic negative | |
| | | | effects on sperm | |
| | | | capacitation and | |
| | | | acrosome reaction. | |

### REFERENCES

1. Maretti C, Cavallini G. The association of a probiotic with a prebiotic (Flortec, Bracco) to improve the quality/quantity of spermatozoa in infertile patients with idiopathic oligoasthenoteratospermia: a pilot study. Andrology. 2017; 5(3):439-44.
2. Valcarce D G, Genoves S, Riesco M F, Martorell P, Herraez M P, Ramon D, et al. Probiotic administration improves sperm quality in asthenozoospermic human donors. Beneficial microbes. 2017; 8(2):193-206.
3. Kolandooz M, Nasri S, Modarres S Z, Kianbakht S, Huseini H F. Effects of Nigella sativa L. seed oil on abnormal semen quality in infertile men: a randomized, double-blind, placebo-controlled clinical trial. Phytomedicine. 2014; 21(6):901-5.
4. Canepa P, Dal Lago A, De Leo C, Gallo M, Rizzo C, Licata E, et al. Combined treatment with myo-inositol, alpha-lipoic acid, folic acid and vitamins significantly improves sperm parameters of sub-fertile men: a multi-centric study. Eur Rev Med Pharmacol Sci. 2018; 22(20):7078-85.
5. Haghighian H K, Haidari F, Mohammadi-Asl J, Dadfar M. Randomized, triple-blind, placebo-controlled clinical trial examining the effects of alpha-lipoic acid supplement on the spermatogram and seminal oxidative stress in infertile men. Fertil Steril. 2015; 104(2):318-24.
6. Durg S, Shivaram S B, Bavage S. Withania somnifera (Indian ginseng) in male infertility: An evidence-based systematic review and meta-analysis. Phytomedicine. 2018; 50:247-56.
7. Shukla K K, Mandi A A, Mishra V, Rajender S, Sankhwar S N, Patel D, et al. Withania somnifera improves semen quality by combating oxidative stress and cell death and improving essential metal concentrations. Reprod Biomed Online. 2011; 22(5):421-7.
8. Ahmad M K, Mandi A A, Shukla K K, Islam N, Rajender S, Madhukar D, et al. Withania somnifera improves semen quality by regulating reproductive hormone levels and oxidative stress in seminal plasma of infertile males. Fertility and Sterility. 2010; 94(3):989-96.
9. Alizadeh F, Javadi M, Karami A A, Gholaminejad F, Kavianpour M, Haghighian H K. Curcumin nanomicelle improves semen parameters, oxidative stress, inflammatory biomarkers, and reproductive hormones in infertile men: A randomized clinical trial. Phytotherapy research: PTR. 2018; 32(3):514-21.
10. Robbins W A, Xun L, FitzGerald L Z, Esguerra S, Henning S M, Carpenter C L. Walnuts improve semen quality in men consuming a Western-style diet: randomized control dietary intervention trial. Biology of reproduction. 2012; 87(4):101.
11. Salas-Huetos A, Moraleda R, Giardina S, Anton E, Blanco J, Salas-Salvado J, et al. Effect of nut consumption on semen quality and functionality in healthy men consuming a Western-style diet: a randomized controlled trial. Am J Clin Nutr. 2018; 108(5):953-62.
12. Filipcikova R, Oborna I, Brezinova J, Novotny J, Wojewodka G, De Sanctis J B, et al. Lycopene improves the distorted ratio between AA/DHA in the seminal plasma of infertile males and increases the likelihood of successful pregnancy. Biomedical papers of the Medical Faculty of the University Palacky, Olomouc, Czechoslovakia. 2015; 159(1):77-82.
13. Noun M, Amani R, Nasr-Esfahani M, Tarrahi M J. The effects of lycopene supplement on the spermatogram and seminal oxidative stress in infertile men: A randomized, double-blind, placebo-controlled clinical trial. Phytotherapy research: PTR. 2019; 33(12):3203-11.
14. Calogero A E, Aversa A, La Vignera S, Corona G, Ferlin A. The use of nutraceuticals in male sexual and reproductive disturbances: position statement from the Italian Society of Andrology and Sexual Medicine (SIAMS). Journal of endocrinological investigation. 2017; 40(12):1389-97.
15. Comhaire F H, El Garem Y, Mahmoud A, Eertmans F, Schoonjans F. Combined conventional/antioxidant “Astaxanthin” treatment for male infertility: a double blind, randomized trial. Asian J Androl. 2005; 7(3):257-62.
16. Smits R M, Mackenzie-Proctor R, Yazdani A, Stankiewicz M T, Jordan V, Showell M G. Antioxidants for male subfertility. Cochrane Database of Systematic Reviews. 2019(3).
17. Wong W Y, Merkus H M, Thomas C M, Menkveld R, Zielhuis G A, Steegers-Theunissen R P. Effects of folic acid and zinc sulfate on male factor subfertility: a double-blind, randomized, placebo-controlled trial. Fertil Steril. 2002; 77(3):491-8.
18. Maleki B H, Tartibian B. Resistance exercise modulates male factor infertility through anti-inflammatory and antioxidative mechanisms in infertile men: A RCT. Life sciences. 2018; 203:150-60.
19. Maleki B H, Tartibian B. High-Intensity Exercise Training for Improving Reproductive Function in Infertile Patients: A Randomized Controlled Trial. Journal of obstetrics and gynaecology Canada: JOGC=Journal d'obstetrique et gynecologie du Canada: JOGC. 2017; 39(7):545-58.
20. Magdi Y, Darwish E, Elbashir S, Majzoub A, Agarwal A. Effect of modifiable lifestyle factors and antioxidant treatment on semen parameters of men with severe oligoasthenoteratozoospermia. Andrologia. 2017; 49(7).
21. Wirleitner B, Vanderzwalmen P, Stecher A, Spitzer D, Schuff M, Schwerda D, et al. Dietary supplementation of antioxidants improves semen quality of IVF patients in terms of motility, sperm count, and nuclear vacuolization. Int J Vitam Nutr Res. 2012; 82(6):391-8.
22. Akmal M, Qadri J Q, Al-Waili N S, Thangal S, Haq A, Saloom K Y. Improvement in human semen quality after oral supplementation of vitamin C. J Med Food. 2006; 9(3):440-2.
23. Smits R M, Mackenzie-Proctor R, Yazdani A, Stankiewicz M T, Jordan V, Showell M G. Antioxidants for male subfertility. Cochrane Database Syst Rev. 2019; 3:Cd007411.
24. Arab A, Hadi A, Moosavian S P, Askari G, Nasirian M. The association between serum vitamin D, fertility and semen quality: A systematic review and meta-analysis. International Journal of Surgery. 2019; 71:101-9.
25. Blomberg Jensen M, Lawaetz J G, Petersen J H, Juul A, Jorgensen N. Effects of Vitamin D Supplementation on Semen Quality, Reproductive Hormones, and Live Birth Rate: A Randomized Clinical Trial. J Clin Endocrinol Metab. 2018; 103(3):870-81.
26. Calogero A E, Gullo G, La Vignera S, Condorelli R A, Vaiarelli A. Myoinositol improves sperm parameters and serum reproductive hormones in patients with idiopathic infertility: a prospective double-blind randomized placebo-controlled study. Andrology. 2015; 3(3):491-5.
27. ElSheikh M G, Hosny M B, Elshenoufy A, Elghamrawi H, Fayad A, Abdelrahman S. Combination of vitamin E and clomiphene citrate in treating patients with idiopathic oligoasthenozoospermia: A prospective, randomized trial. Andrology. 2015; 3(5):864-7.
28. Ghanem H, Shaeer O, El-Segini A. Combination clomiphene citrate and antioxidant therapy for idiopathic male infertility: a randomized controlled trial. Fertil Steril. 2010; 93(7):2232-5.
29. Greco E, Romano S, Iacobelli M, Ferrero S, Baroni E, Minasi M G, et al. ICSI in cases of sperm DNA damage: beneficial effect of oral antioxidant treatment. Hum Reprod. 2005; 20(9):2590-4.
30. Salas-Huetos A, Rosique-Esteban N, Becerra-Tomas N, Vizmanos B, Bullo M, Salas-Salvado J. The Effect of Nutrients and Dietary Supplements on Sperm Quality Parameters: A Systematic Review and Meta-Analysis of Randomized Clinical Trials. Advances in nutrition (Bethesda, Md.). 2018; 9(6):833-48.
31. Gambera L, Stendardi A, Ghelardi C, Fineschi B, Aini R. Effects of antioxidant treatment on seminal parameters in patients undergoing in vitro fertilization. Arch Ital Urol Androl. 2019; 91(3).
32. Jannatifar R, Parivar K, Roodbari N H, Nasr-Esfahani M H. Effects of N-acetyl-cysteine supplementation on sperm quality, chromatin integrity and level of oxidative stress in infertile men. Reprod Biol Endocrinol. 2019; 17(1):24.
33. Agarwal A, Said T M. Carnitines and male infertility. Reprod Biomed Online. 2004; 8(4):376-84.
34. Abad C, Amengual M J, Gosalvez J, Coward K, Hannaoui N, Benet J, et al. Effects of oral antioxidant treatment upon the dynamics of human sperm DNA fragmentation and subpopulations of sperm with highly degraded DNA. Andrologia. 2013; 45(3):211-6.
35. Busetto G M, Agarwal A, Virmani A, Antonini G, Ragonesi G, Del Giudice F, et al. Effect of metabolic and antioxidant supplementation on sperm parameters in oligo-astheno-teratozoospermia, with and without varicocele: A double-blind placebo-controlled study. Andrologia. 2018; 50(3).
36. Moradi M, Moradi A, Alemi M, Ahmadnia H, Abdi H, Ahmadi A, et al. Safety and efficacy of clomiphene citrate and L-carnitine in idiopathic male infertility: a comparative study. Urology journal. 2010; 7(3):188-93.
37. Gvozdjakova A, Kucharska J, Dubravicky J, Mojto V, Singh R B. Coenzyme Q(1)(0), alpha-tocopherol, and oxidative stress could be important metabolic biomarkers of male infertility. Disease markers. 2015; 2015:827941.
38. Lafuente R, Gonzalez-Comadran M, Sola I, Lopez G, Brassesco M, Carreras R, et al. Coenzyme Q10 and male infertility: a meta-analysis. J Assist Reprod Genet. 2013; 30(9):1147-56.
39. Nadjarzadeh A, Shidfar F, Amirjannati N, Vafa M R, Motevalian S A, Gohari M R, et al. Effect of Coenzyme Q10 supplementation on antioxidant enzymes activity and oxidative stress of seminal plasma: a double-blind randomised clinical trial. Andrologia. 2014; 46(2):177-83.
40. Safarinejad M R. The effect of coenzyme Q(1)(0) supplementation on partner pregnancy rate in infertile men with idiopathic oligoasthenoteratozoospermia: an open-label prospective study. International urology and nephrology. 2012; 44(3):689-700.
41. Safarinejad M R, Safarinejad S, Shafiei N, Safarinejad S. Effects of the reduced form of coenzyme Q10 (ubiquinol) on semen parameters in men with idiopathic infertility: a double-blind, placebo controlled, randomized study. J Urol. 2012; 188(2):526-31.
42. Safarinejad M R. Effect of omega-3 polyunsaturated fatty acid supplementation on semen profile and enzymatic anti-oxidant capacity of seminal plasma in infertile men with idiopathic oligoasthenoteratospermia: a double-blind, placebo-controlled, randomised study. Andrologia. 2011; 43(1):38-47.
43. Omu A E, Al-Azemi M K, Kehinde E O, Anim J T, Oriowo M A, Mathew T C. Indications of the mechanisms involved in improved sperm parameters by zinc therapy. Medical principles and practice: international journal of the Kuwait University, Health Science Centre. 2008; 17(2):108-16.
44. Hadwan M H, Almashhedy L A, Alsalman A R S. Study of the effects of oral zinc supplementation on peroxynitrite levels, arginase activity and NO synthase activity in seminal plasma of Iraqi asthenospermic patients. Reproductive biology and endocrinology: RB&E. 2014; 12:1-.
45. Lipovac M, Bodner F, Imhof M, Chedraui P. Comparison of the effect of a combination of eight micronutrients versus a standard mono preparation on sperm parameters. Reprod Biol Endocrinol. 2016; 14(1):84.
46. Safarinejad M R, Safarinejad S. Efficacy of selenium and/or N-acetyl-cysteine for improving semen parameters in infertile men: a double-blind, placebo controlled, randomized study. J Urol. 2009; 181(2):741-51.
47. Dinkova A, Martinov D, Konova E. Efficacy of myo-inositol in the clinical management of patients with asthenozoospermia. Eur Rev Med Pharmacol Sci. 2017; 21(2 Suppl):62-5.
48. Keskes-Ammar L, Feki-Chakroun N, Rebai T, Sahnoun Z, Ghozzi H, Hammami S, et al. Sperm oxidative stress and the effect of an oral vitamin E and selenium supplement on semen quality in infertile men. Archives of andrology. 2003; 49(2):83-94.
49. Afeiche M C, Gaskins A J, Williams P L, Toth T L, Wright D L, Tanrikut C, et al. Processed meat intake is unfavorably and fish intake favorably associated with semen quality indicators among men attending a fertility clinic. The Journal of nutrition. 2014; 144(7):1091-8.
50. Mendiola J, Torres-Cantero A M, Moreno-Grau J M, Ten J, Roca M, Moreno-Grau S, et al. Food intake and its relationship with semen quality: a case-control study. Fertil Steril. 2009; 91(3):812-8.
51. Eslamian G, Amirjannati N, Rashidkhani B, Sadeghi M R, Hekmatdoost A. Intake of food groups and idiopathic asthenozoospermia: a case-control study. Hum Reprod.
52. Afeiche M C, Bridges N D, Williams P L, Gaskins A J, Tanrikut C, Petrozza J C, et al. Dairy intake and semen quality among men attending a fertility clinic. Fertil Steril. 2014; 101(5):1280-7.
53. Gaskins A J, Afeiche M C, Hauser R, Williams P L, Gillman M W, Tanrikut C, et al. Paternal physical and sedentary activities in relation to semen quality and reproductive outcomes among couples from a fertility center. Hum Reprod. 2014; 29(11):2575-82.
54. Braga D P, Halpern G, Figueira Rde C, Setti A S, Iaconelli A, Jr., Borges E, Jr. Food intake and social habits in male patients and its relationship to intracytoplasmic sperm injection outcomes. Fertil Steril. 2012; 97(1):53-9.
55. Efrat M, Stein A, Pinkas H, Unger R, Birk R. Dietary patterns are positively associated with semen quality. Fertil Steril. 2018; 109(5):809-16.
56. Eslamian G, Amirjannati N, Rashidkhani B, Sadeghi M R, Hekmatdoost A. Nutrient patterns and asthenozoospermia: a case-control study. Andrologia. 2017; 49(3).
57. Oostingh E C, Steegers-Theunissen R P, de Vries J H, Laven J S, Koster M P. Strong adherence to a healthy dietary pattern is associated with better semen quality, especially in men with poor semen quality. Fertil Steril. 2017; 107(4):916-23.e2.
58. Ricci E, Bravi F, Noli S, Ferrari S, De Cosmi V, La Vecchia I, et al. Mediterranean diet and the risk of poor semen quality: cross-sectional analysis of men referring to an Italian Fertility Clinic. Andrology. 2019; 7(2):156-62.
59. Salas-Huetos A, Bullo M, Salas-Salvado J. Dietary patterns, foods and nutrients in male fertility parameters and fecundability: a systematic review of observational studies. Hum Reprod Update. 2017; 23(4):371-89.
60. Wise L A, Cramer D W, Hornstein M D, Ashby R K, Missmer S A. Physical activity and semen quality among men attending an infertility clinic. Fertility and Sterility. 2011; 95(3):1025-30.
61. Xia W, Chiu Y H, Williams P L, Gaskins A J, Toth T L, Tanrikut C, et al. Men's meat intake and treatment outcomes among couples undergoing assisted reproduction. Fertil Steril. 2015; 104(4):972-9.
62. Chavarro J E, Toth T L, Sadio S M, Hauser R. Soy food and isoflavone intake in relation to semen quality parameters among men from an infertility clinic. Hum Reprod. 2008; 23(11):2584-90.
63. Xia Y, Chen M, Zhu P, Lu C, Fu G, Zhou X, et al. Urinary phytoestrogen levels related to idiopathic male infertility in Chinese men. Environment international. 2013; 59:161-7.
64. Chavarro J E, Furtado J, Toth T L, Ford J, Keller M, Campos H, et al. Trans-fatty acid levels in sperm are associated with sperm concentration among men from an infertility clinic. Fertility and sterility. 2011; 95(5):1794-7.
65. Eslamian G, Amirjannati N, Rashidkhani B, Sadeghi M R, Baghestani A R, Hekmatdoost A. Dietary fatty acid intakes and asthenozoospermia: a case-control study. Fertil Steril. 2015; 103(1):190-8.
66. Danielewicz A, Przybylowicz K E, Przybylowicz M. Dietary Patterns and Poor Semen Quality Risk in Men: A Cross-Sectional Study. Nutrients. 2018; 10(9).
67. Vujkovic M, de Vries J H, Dohle G R, Bonsel G J, Lindemans J, Macklon N S, et al. Associations between dietary patterns and semen quality in men undergoing IVF/ICSI treatment. Hum Reprod. 2009; 24(6):1304-12.
68. Salas-Huetos A, James E R, Aston K I, Jenkins T G, Carrell D T. Diet and sperm quality: Nutrients, foods and dietary patterns. Reproductive Biology. 2019; 19(3):219-24.
69. Li Y, Lin H, Li Y, Cao J. Association between socio-psycho-behavioral factors and male semen quality: systematic review and meta-analyses. Fertility and Sterility. 2011; 95(1):116-23.
70. Sermondade N, Faure C, Fezeu L, Shayeb A G, Bonde J P, Jensen T K, et al. BMI in relation to sperm count: an updated systematic review and collaborative meta-analysis. Human reproduction update. 2013; 19(3):221-31.

1. A method of enhancing fertility and conception for individuals with sperm conditions comprising:
requesting and receiving a plurality of user attributes;
comparing the plurality of user attributes to a corresponding plurality of evidence-based fertility benchmarks;
determining a plurality of fertility support opportunities based on at least the plurality of user attributes and the comparison to the corresponding plurality of evidence-based fertility benchmarks;
identifying a plurality of fertility enhancing recommendations based on at least the plurality of fertility support opportunities; and
presenting at least one of the plurality of fertility enhancing recommendations.

2. The method according to claim 1 wherein the individuals are considered to have the sperm condition of low sperm count when the sperm count is less than 15 million sperm per milliliter measured in a semen sample.

3. The method according to claim 1 wherein the individuals are considered to have the sperm condition of low sperm motility when sperm motility is less than 40% sperm motility or less than 32% progressive sperm motility.

4. The method according to claim 1 wherein the individuals are considered to have the sperm-related condition of abnormal sperm morphology when sperm are less than 30% morphologically normal sperm.

5. The method according to claim 1 wherein the individuals are considered to have the sperm-related condition of low sperm concentration when the sperm concentration is less than 20×106/mL concentration in semen.

6. The method according to claim 1 wherein the individuals are considered to have the sperm-related condition according to any one of claims 2 to 5 which is associated with low testosterone below 270 to 1,070 nanograms per deciliter (ng/dL).

7. The method according to claim 1 wherein the sperm-related condition is associated with an individual who is obese with a BMI 30.0 or greater.

8-9. (canceled)

10. The method according to claim 1 wherein the identifying of a plurality of fertility enhancing recommendations includes the dietary recommendations.

11. (canceled)

12. The method according to claim 1 comprising recommendations for supplements selected from the group consisting of:
(i) Vitamin E;
(ii) Zinc;
(iii) N-acetyl cysteine;
(iv) L-acetyl carnitine;
(v) Omega fatty acids selected from DHA and EPA;
(vi) Alpha-lipoic acid;
(vii) Selenium;
(viii) Coenzyme Q;
(ix) Folic acid;
(x) Vitamin A;
(xi) Lycopene;
(xii) Beta carotene;
(xiii) Vitamin C;
(xiv) Vitamin D;
(xv) Myoinositol;
(xvi) Nigella sativa seed oil;
(xvii) Withania somnifera;
(xviii) Curcumin; and
(xix) Glutathione;

13-21. (canceled)

22. The method according to claim 1 wherein the identifying of a plurality of fertility enhancing recommendations includes the food recommendation that the low fat dairy food comprises low fat milk in the amount of at least 1 serving per day.

23. The method according to claim 1 wherein the identifying of a plurality of fertility enhancing recommendations includes diet recommendations selected from the group consisting of:
(i) avoidance of processed meats;
(ii) avoidance of full fat dairy products and cheese;
(iii) avoidance of sweets;
(iv) avoidance of trans-fats and saturated fats;
(v) caloric restriction with and without high protein in obese men with a BMI over 25 kg/m2, more preferably with a BMI over 30 kg/m2;
(vi) reducing alcoholic beverages and sugar sweetened beverages to less than one per day; and
(vii) consuming soy-based products in moderation.

24. (canceled)

25. The method according to claim 1 wherein the identifying of a plurality of fertility enhancing recommendations based on at least the plurality of fertility support opportunities comprises:
providing the plurality of fertility support opportunities to a cloud-based artificial intelligence service; and
receiving, from the cloud-based artificial intelligence service, a plurality of fertility enhancing recommendations based on the fertility support opportunities provided to the cloud-based artificial intelligence service.

26. A computer implemented system for generating fertility enhancing recommendations for enhancing fertility and conception for individuals with sperm conditions comprising:
a memory;
a CPU;
a display configured to present an attribute questionnaire to a user;
an attribute receiving unit configured to receive a plurality of user attributes based on at least the attribute questionnaire;
an attribute comparison unit configured to compare the received plurality of user attributes to a corresponding plurality of fertility attribute benchmarks;
an evidence-based diet and lifestyle recommendation engine configured to generate a plurality of fertility enhancing recommendations based on at least the plurality of user attributes and the comparison to corresponding plurality of fertility attribute benchmarks; and
wherein the display is further configured to present at least one of the plurality of the fertility enhancing recommendations to the user.

27. The system of claim 26, wherein the plurality of user attributes comprises information regarding the individual with sperm conditions selected from the group comprising:
(i) low sperm count, wherein low sperm count is less than 15 million sperm per milliliter measured in a semen sample;
(ii) low sperm motility with less than 40% sperm motility or less than 32% with progressive sperm motility;
(iii) abnormal sperm morphology with less than 30% morphologically normal sperm;
(iv) low sperm concentration with less than 20×106/mL concentration in semen;
(v) sperm-related condition associated with low testosterone below 270 to 1,070 nanograms per deciliter (ng/dL); or
(vi) sperm-related condition associated obesity with a BMI 30.0 or greater.

28. The system according to claim 26 wherein the plurality of user attributes comprises information regarding at least one further user attribute selected from the group of: age, gender, weight, height, activity level, food sensitivities, preferred diet, fertility status, lifestyle choices, and any co-morbidities.

29. (canceled)

30. The system according to claim 26, wherein the evidence-based diet and lifestyle recommendation engine comprises:
a plurality of databases comprising one or more of recipes, specific food items, products, or diet tips;
a plurality of filters by dietary restrictions comprising one or more of food sensitivities, preferred diets, fertility-related conditions, or co-morbidities; and
an optimization unit configured to optimize the plurality of fertility enhancing recommendations based on one or more of caloric intake, food groups, or nutrients.

31. The system according to claim 26 wherein the optimization unit is configured to optimize fertility enhancing recommendations including recommendations of dietary supplements for individuals with sperm conditions, comprising dietary supplements selected from the group consisting of:
(i) Vitamin E;
(ii) Zinc;
(iii) N-acetyl cysteine;
(iv) L-acetyl carnitine;
(v) Omega fatty acids selected from DHA and EPA;
(vi) Alpha-lipoic acid;
(vii) Selenium;
(viii) Coenzyme Q;
(ix) Folic acid;
(x) Vitamin A;
(xi) Lycopene;
(xii) Beta carotene;
(xiii) Vitamin C;
(xiv) Vitamin D;
(xv) Myoinositol;
(xvi) Nigella sativa seed oil;
(xvii) Withania somnifera;
(xviii) Curcumin; and
(xix) Glutathione;

32. The system of claim 26 wherein the optimization unit is configured to optimize fertility enhancing recommendations including recommendations of dietary supplements for individuals with sperm conditions, comprising supplements administered in the following dosing amounts as separate supplements or in combination selected from the group consisting of:
(i) Vitamin E in the amount of 20 to 100 mg/day for 2 to 12 months;
(ii) Zinc in the amount of 66 to 500 mg/day for 3 to 6 months;
(iii) N-acetyl cysteine in the amount of at least about 600 mg/day;
(iv) L-acetyl carnitine in the amount of 1000 to 3000 mg/day;
(v) Omega fatty acids selected from DHA in the amount of 400 to 1120 mg/day and EPA in the amount of 135 to 720 mg/day for 12 to 32 weeks;
(vi) Alpha-lipoic acid in the amount of 600 mg/day for at least 12 weeks;
(vii) Selenium in the amount of 100 to 200 ug/day;
(viii) Coenzyme Q in the amount of 100 to 300 mg/day;
(ix) Folic acid in the amount of 200 ug to 5 mg/day for at least 26 weeks;
(x) Vitamin A in the amount of 1 to 30 mg/day;
(xi) Lycopene in the amount of 6 to 8 mg/day;
(xii) Beta carotene in the amount of 18 to 30 mg/day;
(xiii) Vitamin C in the amount of 500 to 1000 mg/day for 3 to 12 weeks;
(xiv) Vitamin D in the amount of at least 15 ug/day;
(xv) Myoinositol in the amount of at least about 4 g/day for 2 to 3 months;
(xvi) Nigella sativa seed oil in the amount of at least about 5 ml/day or 5 mg/day;
(xvii) Withania somnifera in the amount of at least 5 g/day for at least 3 months;
(xviii) Curcumin in the amount of at least about 80 mg/day for at least 10 weeks; and
(xix) Glutathione in the amount of at least about 600 mg/day.

33-35. (canceled)
